(19) United States
(12) Patent Application Publication
(10) Pub. No.: US 2025/0228928 A1
(43) Pub. Date: Jul. 17, 2025

(54) IMMUNE ENHANCEMENT AND INFECTIOUS DISEASE TREATMENT

(71) Applicant: Spark Therapeutics, Inc., Philadelphia, PA (US)

(72) Inventors: Xavier ANGUELA, Barcelona (ES); Sean ARMOUR, Holland, PA (US); Pedro CEJAS, Oreland, PA (US); Ali NAHVI, Needham, MA (US); Seoyun YUM, Philadelphia, PA (US); Rui ZHANG, Winchester, MA (US)

(21) Appl. No.: 18/853,434

(22) PCT Filed: Mar. 31, 2023
(86) PCT No.: PCT/US2023/017097

§ 371 Date: Oct. 2, 2024

Related U.S. Application Data
(60) Provisional application No. 63/362,450, filed on Apr. 4, 2022.

(51) Int. Cl. A61K 39/215; A61K 9/51; A61K 39/00; A61K 39/23; A61K 39/385; A61K 39/395; A61K 45/06

(52) U.S. Cl. CPC A61K 39/215; A61K 9/5123; A61K 39/23; A61K 39/385; A61K 39/3955; A61K 45/06; A61K 2039/53; A61K 2039/6087

(57) ABSTRACT

The present invention features methods utilizing nanoparticles for double-stranded DNA (dsDNA). The nanoparticles are able to deliver the dsDNA intracellularly where the dsDNA can stimulate the innate immune response. Uses of the described methods include enhancing an immune response to a vaccine and infectious disease treatment.

### FIELD OF THE INVENTION

The invention relates to methods for enhancing an immune response and methods of treating infectious disease. The provided methods can be used, for example, in combination with different types of therapeutic agents targeting infectious disease and with vaccines.

### BACKGROUND OF THE INVENTION

Infectious diseases are disorders caused by pathogens such as bacteria, viruses, fungi or parasites. Infectious diseases are major causes of mortality around the world and pose significant health, social and economic burdens. Lower respiratory infections, diarrheal disease and tuberculosis are among the top 10 causes of death. (Gu et al., Frontiers in Microbiology (2021) volume 12, article 667561; and van Seventer and Hochberg International Encyclopedia of Public Health. (2017) 2^{nd} edition 6:22-39.)

Methods of treatment for disease causing infectious organisms (also referred to herein as pathogens) include both prophylactic treatment and therapeutic treatment. Prophylactic treatment involves treating a subject prior to pathogen infection. Prophylactic treatment can be performed, for example, on the general population, a person at a higher risk of being infected, or a group of people at greater risk of infection. Prophylactic treatment includes the use of a vaccine boosting a host immune response against a pathogen; and the use of a therapeutic agent that can boost one or more components of a host immune response and/or target a particular pathogen.

Therapeutic treatment involves treating a person infected with a pathogen. Therapeutic treatment can be performed, for example, through the use of a therapeutic vaccine boosting a host immune response against a pathogen; and through the use of a therapeutic agent that can boost one or more components of the host immune system and/or target a particular pathogen.

Examples of different types of vaccines including therapeutic vaccines are provided in Kutsher et al., (2012) Microbial Biotechnology 5(2), 270-282 and Plotkin's Vaccine 7^{th} Edition (2018) Edited by Plotkin et al.

### BRIEF SUMMARY OF THE INVENTION

The present invention features methods utilizing nanoparticles for delivering a double-stranded DNA (dsDNA). The nanoparticles are able to deliver the dsDNA intracellularly where the dsDNA can stimulate the innate immune response. The featured methods may include use of therapeutic agents targeting infectious disease and boosting the immune system; and vaccines producing an immune against an infectious organism.

Thus, a first aspect of the present invention describes a method of treatment in a subject for an infectious disease, comprising administering to the subject: (a) a nanoparticle comprising a dsDNA; and (b) a vaccine or a therapeutic agent. In an embodiment, the dsDNA comprises a dsDNA region at least 45 base pairs in length.

Reference to “treating” and “treatment” includes prophylactic treatment and/or treating a subject having an infectious disease.

A “vaccine” provides an antigen to which an immune response is directed. A variety of different vaccines can be used including live attenuated, killed whole organism, toxoid, subunit (e.g., purified protein, recombinant protein, polysaccharide, and peptide), virus-like particle, outer membrane vesicle, protein-polysaccharide conjugate, viral vector, nucleic acid, bacterial vectored, and antigen presenting cells.

A “therapeutic agent” provides a compound having activity against an infectious disease and/or boosts one or more components of the host immune response against an infectious organism. Reference to compound or agent is not a limitation as to the size or complexity of the compound or agent. Examples of compounds and agents include small molecules and larger molecules such as antibodies and other proteins.

Another aspect of the present invention describes a method for enhancing an immune response to a vaccine in a subject. The method comprises administering to the subject (a) a nanoparticle comprising a dsDNA and (b) a vaccine. In an embodiment, the dsDNA comprises a dsDNA region at least 45 base pairs in length.

Additional aspects are directed to a nanoparticle comprising a dsDNA, for use in the methods described herein; and use of a nanoparticle comprising a dsDNA, for the preparation of a medicament, preferably for a method described herein. In an embodiment, the dsDNA comprises a dsDNA region at least 45 base pairs in length.

Other features and advantages of the present invention are apparent from additional descriptions provided herein, including different examples. The provided examples illustrate different components and methodology useful in practicing the present invention. Such examples do not limit the claimed invention. Based on the present disclosure, the skilled artisan can identify and employ other components and methodology useful for practicing the present invention.

### BRIEF DESCRIPTION OF THE DRAWINGS

FIGS. 1A and 1B illustrate cytokine response in mice 6 hours post-dosing with SARS-CoV-2 receptor protein (RBD), DNA-LNP (labeled “DNA-NP”), and different adjuvants (Adju-Phos and AddaVax). FIG. 1A shows serum IFN-γ levels and FIG. 1B shows serum IL-6 levels. “LLOQ” indicates lower limit of quantification.

FIG. 2 illustrates anti-RBD IgG antibody titers in mice at day 38 post-dosing with SARS-CoV-2 virus receptor protein (RBD), DNA-LNP (labeled “DNA-NP”), and different adjuvants (Adju-Phos and AddaVax). “ULOD” indicates upper limit of detection. “LLOD” indicates lower limit of detection.

FIG. 3A and FIG. 3B illustrate anti-RBD IgG subtypes and the ratios thereof, respectively, in mice at day 38 post-dosing with SARS-CoV-2 virus receptor protein (RBD), DNA-LNP (labeled “DNA-NP”), and different adjuvants (Adju-Phos and AddaVax). “ULOD” indicates upper limit of detection. “LLOD” indicates lower limit of detection.

FIG. 4 illustrates anti-AAV antibody levels in plasma two weeks after dosing with AAV, AAV mixed with “empty” (no DNA) LNP, and AAV adjuvanted with DNA-LNP. Reference to *** indicates a P-value <0.001, and reference to **** indicates a P-value <0.0001.

### DETAILED DESCRIPTION OF THE INVENTION

The present invention features methods utilizing nanoparticles for delivering a dsDNA for treatment of infectious disease and immune enhancement. As illustrated in the Examples below, the dsDNA can stimulate the innate immune response and provide adjuvant activity. While not wishing to be bound by any theory, the nanoparticles facilitate intracellular delivery of the dsDNA, where the dsDNA stimulates the innate immune response of the cytosolic sensing cGAS/STING and/or inflammasome pathways to provide an immune response.

Method for treatment of infectious disease involves using one or more therapeutic agent having anti-infectious disease activity and/or a vaccine providing an infectious organism antigen. Therapeutic agents can, for example, directly target an infectious organisms and/or boost one or component of the host immune system.

Reference to “dsDNA” provides one or more polynucleotides that forms one or more double-stranded DNA regions. A dsDNA region may be formed from a single polynucleotide or two different polynucleotides. dsDNA may contain modified nucleotides, for example, sugar modifications (e.g., 2′-methoxyethyl (2′-MOE), 2′-fluor (2′-F), locked nucleic acid (LNA), constrained ethyl (cEt) and tricyclo-DNA (tc-DNA)), base modifications (e.g., C7-modified deaza-adenine (e.g., methyl, Cl or F), C7-modified deaza-guanosine (e.g., methyl, Cl or F), C5-modified cytosine (e.g., methyl, F or Cl), and C5-modified uridine (e.g., methyl, F or Cl), and/or backbone modifications (e.g., phosphorothioate (Rp and/or Rs), thio-phosphoramidate, phosphorodiamidate morpholino oligos (PMO), and peptide-nucleic acid (PNA)). Examples of modified nucleotides are provided in, for example, Adachi et al., (2021) Biomedicines 9, 550; Shen and Corey (2018) Nucleic Acid Research 46: 4, 1584-1600; and Duffy et al., (2020) 18:112; each of with are hereby incorporated by reference herein in their entirety.

Reference to “polynucleotide” provides a nucleic acid polymer made up of naturally occurring nucleotides and/or modified nucleotides. Nucleotides may contain sugar modifications (e.g., 2′-methoxyethyl (2′-MOE), 2′-fluor (2′-F), locked nucleic acid (LNA), constrained ethyl (cEt) and tricyclo-DNA (tc-DNA)), base modifications (e.g., C7-modified deaza-adenine (e.g., methyl, Cl or F), C7-modified deaza-guanosine (e.g., methyl, Cl or F), C5-modified cytosine (e.g., methyl, F or Cl), and C5-modified uridine (e.g., methyl, F or Cl)), and/or backbone modifications (e.g., phosphorothioate (Rp and/or Rs), thio-phosphoramidate, phosphorodiamidate morpholino oligos (PMO), and peptide-nucleic acid (PNA)).

dsDNA comprises a dsDNA region and may also comprise additional regions. Examples of additional regions include single-stranded regions, RNA regions, modified RNA modified DNA regions and regions that are not nucleotides. In certain embodiments the dsDNA comprises a continuous polynucleotide strand providing a structure with a dsDNA region (e.g., a hair-pin loop) or comprises two polynucleotide strands where all or a region of the two strands form the dsDNA region. In different embodiments, the dsDNA is a minicircle, a nanoplasmid, open linear duplex DNA, and closed-ended linear duplex DNA (CELiD/ceDNA/doggybone DNA).

dsDNA can be produced using different techniques including enzymatic production of nucleotide polymers and/or chemical modification. Examples of techniques for producing nucleic acid are well known in the art and include, for example: Kosuri et al., (2014) Nat. Methods. 11(5):499-507; Ducani et al., (2013) Nat. Methods 10, 647-652; Ducani et al., (2014) Nucleic Acids Research, Volume 42, Issue 16; and Sandahl et al., (2021) Nat. Commun. 12, 2760.

In certain embodiments, nucleotide modifications do not significantly decrease the ability of the dsDNA to stimulate the innate immune response. In different embodiments, the dsDNA is able to stimulate an innate immune response of at least 50%, at least 65%, at least 75%, at least 85%, at least 90%, or least 100% compared to the corresponding unmodified dsDNA as measured by IFN-γ and IL-6 as provided in the Examples below.

A “nanoparticle” refers to a small non-viral particle that can encapsulate or associate with dsDNA and facilitates dsDNA delivery to a cell. Examples of nanoparticles include lipid nanoparticles (LNP), polymeric nanoparticles, lipid polymer nanoparticles (LPNP), protein and peptide-based nanoparticles, DNA dendrimers and DNA-based nanocarriers, carbon nanotubes, microparticles, microcapsules, inorganic nanoparticles, peptide cage nanoparticles, and exosomes. The nanoparticle ranges in size from about 10 nm to about 1000 nm. In different embodiments, the nanoparticle is about 50 nm to about 500 nm, or about 50 nm to about 200 nm.

Reference to “subject” indicates a mammal, including humans; non-human primates such as apes, gibbons, gorillas, chimpanzees, orangutans, macaques; domestic animals, such as dogs and cats; farm animals such as poultry and ducks, horses, cows, goats, sheep, and pigs; and experimental animals such as mice, rats, rabbits, and guinea pigs. A preferred subject is a human subject.

A DNA vector contains a transgene operative linked to one or more regulatory element providing for RNA expression from the transgene. The produced RNA can itself be functional or can encode for a protein. One type of regulatory element is a promoter, which binds RNA polymerase and the necessary transcription factors to initiate transcription. When encoding for protein, the produced RNA sequence will also encode a termination sequence at the end of the coding sequence.

The term “operatively linked” refers to the association of two or more nucleic acid segments on a single nucleic acid where the function of one is affected by the other.

Reference to “transgene” indicates a DNA region capable of being expressed to RNA, without regard to origin of the polynucleotide sequence. The transgene is generally part of a longer length nucleic acid, where the nucleic acid contains at least one region with which the transgene is not normally associated with in nature.

The singular forms “a,” “an,” and “the” include plural reference unless the context clearly dictates otherwise.

As used herein, the conjunctive term “and/or” between multiple recited elements is understood to encompass both individual and combined options. For instance, where two elements are conjoined by “and/or”, a first option refers to the applicability of the first option without the second, a second option refers to the applicability of the second option without the first, and a third option refers to the applicability of the first and second options together. Any one of the options is understood to fall within the meaning, and therefore satisfy the requirement of the term “and/or”. Concurrent applicability of more than one of the options is also understood to fall within the meaning of the term “and/or.”

Unless clearly indicated otherwise by the context employed the terms “or” and “and” have the same meaning as “and/or”.

Reference to terms such as “including”, “for example”, “e.g.,”, “such as” followed by different members or examples, are open-ended descriptions where the listed members or examples are illustrative and other member or examples can be provided or used.

The terms “polypeptides,” “proteins” and “peptides” can be used interchangeably to refer to an amino acid sequence without regard to function. Polypeptides and peptides contain at least two amino acids, while proteins contain at least about 50 amino acid acids. The provided amino acids include naturally occurring amino acids and modified amino acids such as those provided by cellular modification.

Reference to “comprise”, and variations such as “comprises” and “comprising”, used with respect to an element or group of elements is open-ended and does not exclude additional unrecited elements or method steps. Terms such as “including”, “containing” and “characterized by” are synonymous with comprising. In the different aspects and embodiments described herein, reference to an open-ended term such as “comprising” can be replaced by the terms “consisting” or “consisting essentially of”.

Reference to “consisting of” excludes any element, step, or ingredient not specified in the listed claim elements, where such element, step or ingredient is related to the claimed invention.

Reference to “consisting essentially of” limits the scope of a claim to the specified materials or steps and those that do not materially affect the basic and novel characteristic(s) of the claimed invention.

The term “about” refers to a value within 10% of the underlying parameter (i.e., plus or minus 10%). For example, “about 1:10” includes 1.1:10.1 or 0.9:9.9, and “about 5 hours” includes 4.5 hours or 5.5 hours. The term “about” at the beginning of a string of values modifies each of the values by 10%.

All numerical values or numerical ranges include integers within such ranges and fractions of the values or the integers within ranges unless the context clearly indicates otherwise. Thus, to illustrate, reference to reduction of 95% or more includes 95%, 96%, 97%, 98%, 99%, 100%, as well as 95.1%, 95.2%, 95.3%, 95.4%, 95.5%, etc., 96.1%, 96.2%, 96.3%, 96.4%, 96.5% and so forth; reference to a numerical range, such as “1-4” includes 2, 3, as well as 1.1, 1.2, 1.3, 1.4 and so forth; reference to “1 to 4 weeks” includes 7, 8, 9, 10, 11, 12, 13, 14, 15, 16, 17, 18, 19, 20, 21, 22, 23, 24, 25, 26, 27, or 28 days.

Reference to an integer with more (greater) or less than includes numbers greater or less than the reference number, respectively. Thus, for example, reference to more than 2 includes 3, 4, 5, 6, 7, 8, 9, 10, 11, 12, 13, 14, 15; and administration “two or more” times includes 2, 3, 4, 5, 6, 7, 8, 9, 10, 11, 12, 13, 14, 15, or more times.

Various references including articles and patent publications are cited or described in the background and throughout the specification. Each of these references is herein incorporated by reference in their entirety. None of the references are admitted to be prior art with respect to any inventions disclosed or claimed. In some cases, particular references are indicated to be incorporated by reference herein to highlight the incorporation.

The definitions provided herein, including those in the present section and other sections of the application apply throughout the present application.

Unless defined otherwise, all technical and scientific terms used herein have the same meaning commonly understood to one of ordinary skill in the art to which this invention pertains.

The description has been separated into various sections and paragraphs, and provides various embodiments. These separations should not be considered as disconnecting the substance of a paragraph or section or embodiments from the substance of another paragraph or section or embodiment. The provided descriptions have broad application and encompasses all the combinations of the various sections, paragraphs and sentences that can be contemplated. The discussion of any embodiment is meant only to be exemplary and is not intended to suggest the scope of the disclosure, including the claims (unless otherwise provided in the claims), is limited to these examples.

The instant invention is generally disclosed herein using affirmative language to describe the numerous embodiments of the instant invention. The instant invention also specifically includes embodiments in which particular subject matter is excluded, in full or in part, such as substances or materials, method steps and conditions, protocols, or procedures. For example, in certain embodiments of the instant invention, materials and/or method steps are excluded. Thus, even though the instant invention is generally not expressed herein in terms of what the instant invention does not include, aspects that are not expressly excluded in the instant invention are nevertheless disclosed herein.

### I. Nanoparticles

A variety of different nanoparticles can be employed including lipid nanoparticles (LNP), polymeric nanoparticles, lipid polymer nanoparticles (LPNP), protein and peptide-based nanoparticles, DNA dendrimers and DNA-based nanocarriers, carbon nanotubes, microparticles, microcapsules, inorganic nanoparticles, peptide cage nanoparticles, and exosomes. (See, e.g., Riley and Vermerris Nanomaterials (2017) 201, 7, 94; Thomas et al., Molecules (2019), 24, 3744; Bochicchio et al., (2021), 13, 198; Munagala et al., Cancer Letters (2021), 505, 58; Fu et al., (2020) Nanolmpact 20, 100261; and Neshat et al. (2020) Current Opin. Biotechnol. 66:1-10.)

If desired, a nanoparticle can target a cell type using, for example, targeting ligands recognizing a target cell receptor. Examples of targeting ligands include carbohydrates (e.g., galactose, mannose, glucose, and galactomannan), endogenous ligands (e.g., folic acid and transferrin), antibodies (e.g., anti-HER2 antibody and hD1) and protein/peptides (e.g., RGD, epidermal growth factor, and low density lipoprotein) and peptides. (For example, Teo et al., Advanced Drug Delivery Reviews (2016), 98, 41.)

The present application features the use of nanoparticles to deliver dsDNA. In different embodiments, nanoparticles can deliver additional therapeutic compounds; one or more additional compounds is provided in different nanoparticles; and one or more additional compounds is provided in the same nanoparticle as the dsDNA. Reference to compounds include small molecules and large molecules (e.g., therapeutic proteins and antibodies).

The production of different nanoparticles and incorporation of nucleic acid and other compounds is well known in the art, and exemplified by different publications throughout the discussion in Section I. Examples of publications illustrating incorporation of nucleic acid in a particular nanoparticle such as an LPNP and a LNP include Teo el al., Advanced Drug Delivery Reviews (2016) 98, 41; Bochicchio et al., Pharmaceutics (2021) 13, 198; Mahzabin and Das, IJPSR (2021) 12(1), 65; and Teixeira et al., (2017) Prog. Lipid Res. October; 68:1-11 (each of which are hereby incorporated by reference herein in their entirety). Factors that may impact small molecule incorporation into a nanoparticle include hydrophobicity and the presence of an ionizable moiety. (See, e.g., Nii and Ishii International Journal of Pharmaceutics (2005) 298, 198; and Chen et al., Journal of Controlled Release (2018) 286, 46.)

### I.A. Lipid-Based Delivery Systems

Lipid-based delivery systems include the use of a lipid as a component. Examples of lipid-based delivery systems include liposomes, lipid nanoparticles, micelles, and extracellular vesicles. In certain embodiments, the lipid nanoparticle comprises one or more internal ordered lipid structures, as opposed to, for example a liposome that comprises a complete lipid bilayer and an aqueous core.

A “lipid nanoparticle” or “LNP” refers to a lipid-based vesicle useful for delivery of nucleic acid molecules and having dimensions on the nanoscale. In different embodiments, the nanoparticle is from about 10 nm to about 1000 nm, about 50 nm to about 500 nm, or about 50 nm to about 200 nm.

DNA is negatively charged. Thus, it can be beneficial for the LNP to comprise a cationic lipid such as, for example, an amino lipid. Exemplary amino lipids are described in U.S. Pat. Nos. 9,352,042, 9,220,683, 9,186,325, 9,139,554, 9,126,966 9,018,187, 8,999,351, 8,722,082, 8,642,076, 8,569,256, 8,466,122, and 7,745,651 and U.S. Patent Publication Nos. 2016/0213785, 2016/0199485, 2015/0265708, 2014/0288146, 2013/0123338, 2013/0116307, 2013/0064894, 2012/0172411, and 2010/0117125, all of which are incorporated herein in their entirety. In certain embodiments, the LNP comprises amino lipids such as any of those described in WO2013/063468, hereby incorporated herein in its entirety.

The terms “cationic lipid” and “amino lipid” are used interchangeably herein to include lipids and salts thereof having one, two, three, or more fatty acid or fatty alkyl chains and a pH-titratable amino group (e.g., an alkylamino or dialkylamino group). The cationic lipid is typically protonated (i.e., positively charged) at a pH below the pKa of the cationic lipid and is substantially neutral at a pH above the pKa. The cationic lipid can also be titratable cationic lipids. In certain embodiments, the cationic lipids comprise a protonatable tertiary amine (e.g., pH-titratable) group; C18 alkyl chains, wherein each alkyl chain independently can have one or more double bonds, one or more triple bonds; and ether, ester, or ketal linkages between the head group and alkyl chains.

Cationic lipids include 1,2-dilinoleyloxy-N,N-dimethylaminopropane (DLinDMA), 1,2-dilinolenyloxy-N,N-dimethylaminopropane (DLenDMA), 1,2-di-γ-linolenyloxy-N,N-dimethylaminopropane (γ-DLenDMA), 2,2-dilinoleyl-4-(2-dimethylaminoethyl)-[1,3]-dioxolane (DLin-K-C2-DMA, also known as DLin-C2K-DMA, XTC2, and C2K), 2,2-dilinoleyl-4-dimethylaminomethyl-[1,3]-dioxolane (DLin-K-DMA), dilinoleylmethyl-3-dimethylaminopropionate (DLin-M-C2-DMA, also known as MC2), (6Z,9Z,28Z,31 Z)-heptatriaconta-6,9,28,31-tetraen-19-yl 4-(dimethylamino)butanoate (DLin-M-C3-DMA, also known as MC3), salts thereof, and mixtures thereof. Other cationic lipids also include 1,2-distearyloxy-N,N-dimethyl-3-aminopropane (DSDMA), 1,2-dioleyloxy-N,N-dimethyl-3-aminopropane (DODMA), 2,2-dilinoleyl-4-(3-dimethylaminopropyl)-[1,3]-dioxolane (DLin-K-C3-DMA), 2,2-dilinoleyl-4-(3-dimethylaminobutyl)-[1,3]-dioxolane (DLin-K-C4-DMA), DLen-C2K-DMA, γ-DLen-C2K-DMA, and (DLin-MP-DMA) (also known as 1-B11).

Still further cationic lipids include 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N-(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), dexamethasone-sperimine (DS) and disubstituted spermine (D2S) or mixtures thereof.

A number of commercial preparations of cationic lipids can be used, such as, LIPOFECTIN® (including DOTMA and DOPE, available from GIBCO/BRL), and LIPOFECTAMINE® (comprising DOSPA and DOPE, available from GIBCO/BRL).

Additional ionizable lipids that can be used include C12-200, 306Oi10, MC3, CKK-E12, Lipid 5, Lipid 9, ATX-002, ATX-003, and Merck-32. US Patent Publication No. 2017/0367988, describes Merck-32.

In further embodiments, cationic lipid can be present in an amount from about 10% by molar ratio of the LNP to about 85% by molar ratio of the LNP, or from about 50% by molar ratio of the LNP to about 75% by molar ratio of the LNP.

LNP can comprise a neutral lipid. Neutral lipids can comprise lipid species existing either in an uncharged or neutral zwitterionic form at physiological pH. Such lipids include diacylphosphatidylcholine, diacylphosphatidylethanolamine, ceramide, sphingomyelin, dihydrosphingomyelin, cephalin, and cerebrosides. The selection of neutral lipids is generally guided by considerations including particle size and stability. In certain embodiments, the neutral lipid component can be a lipid having two acyl groups (e.g., diacylphosphatidylcholine and diacylphosphatidylethanolamine).

Lipids having a variety of acyl chain groups of varying chain length and degree of saturation are available or can be isolated or synthesized. In certain embodiments, lipids containing saturated fatty acids with carbon chain lengths in the range of C14 to C22 can be used. In certain embodiments lipids with mono or di-unsaturated fatty acids with carbon chain lengths in the range of C14 to C22 are used. Additionally, lipids having mixtures of saturated and unsaturated fatty acid chains can be used. Exemplary neutral lipids include 1,2-dioleoyl-sn-glycero-3-phosphatidyl-ethanolamine (DOPE), 1,2-distearoyl-sn-glycero-3-phosphocholine (DSPC), 1-palmitoyl-2-oleoyl-sn-glycero-3-phosphocholine (POPC), or a phosphatidylcholine. The neutral lipids can also be composed of sphingomyelin, dihydrosphingomyelin, or phospholipids with other head groups, such as serine and inositol.

In further embodiments providing for neutral lipids, the neutral lipid can be present in an amount from about 0.1% by weight of the lipid nanoparticle to about 99% by weight of the LNP, or from about 5% by weight of the LNP to about 15% by weight of the LNP, e.g., about 1%, about 2%, about 3%, about 4%, about 5%, about 6%, about 7%, about 8%, about 9%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 99%.

LNP can be combined with additional components such as sterols and polyethylene glycol. Sterols can confer fluidity to the LNP. As used herein “sterol” refers to naturally occurring sterol of plant (phytosterols) or animal (zoosterols) origin as well as non-naturally occurring synthetic sterols, all of which are characterized by the presence of a hydroxyl group at the 3-position of the steroid A-ring. Suitable sterols include those conventionally used in the field of liposome, lipid vesicle or lipid particle preparation, most commonly cholesterol. Phytosterols include campesterol, sitosterol, and stigmasterol. Sterols also include sterol-modified lipids, such as those described in U.S. Patent Application Publication 2011/0177156. In different embodiments providing for a sterol, the sterol is present in an amount from about 1% by weight of the LNP to about 80% by weight of the LNP or from about 10% by weight of the LNP to about 25% by weight of the LNP.

Polyethylene glycol (PEG) is a linear, water-soluble polymer of ethylene PEG repeating units with two terminal hydroxyl groups. PEGs are classified by their molecular weights, for example, PEG 2000 has an average molecular weight of about 2,000 daltons, and PEG 5000 has an average molecular weight of about 5,000 daltons. PEGs commercially available from Sigma Chemical Co. and other companies and include monomethoxypolyethylene glycol (MePEG-OH), monomethoxypolyethylene glycol-succinate (MePEG-S), monomethoxypolyethylene glycol-succinimidyl succinate (MePEG-S-NHS), monomethoxypolyethylene glycol-amine (MePEG-NH2), monomethoxypolyethylene glycol-tresylate (MePEG-TRES), and monomethoxypolyethylene glycol-imidazolyl-carbonyl (MePEG-IM).

In certain embodiments concerning PEG, PEG has an average molecular weight of about 550 to about 10,000 daltons and is optionally substituted by alkyl, alkoxy, acyl or aryl. In further embodiments, the PEG can be substituted with methyl at the terminal hydroxyl position. In further embodiments, the PEG can have an average molecular weight from about 750 to about 5,000 daltons, or from about 1,000 to about 5,000 daltons, or from about 1,500 to about 3,000 daltons or from about 2,000 daltons or from about 750 daltons.

PEG-modified lipids include the PEG-dialkyloxypropyl conjugates (PEG-DAA) described in U.S. Pat. Nos. 8,936,942 and 7,803,397. PEG-modified lipids (or lipid-polyoxyethylene conjugates) can have a variety of “anchoring” lipid portions to secure the PEG portion to the surface of the lipid vesicle. Examples of suitable PEG-modified lipids include PEG-modified phosphatidylethanolamine and phosphatidic acid, PEG-ceramide conjugates (e.g., PEG-CerC14 or PEG-CerC20) which are described in U.S. Pat. No. 5,820,873, PEG-modified dialkylamines and PEG-modified 1,2-diacyloxypropan-3-amines. In certain embodiments, the PEG-modified lipid can be PEG-modified diacylglycerols and dialkylglycerols. In certain embodiments, the PEG can be in an amount from about 0.1% by weight of the LNP to about 50% by weight of the LNP, or from about 5% by weight of the LNP to about 15% by weight of the LNP.

In further embodiments concerning LNP size, prior to encapsulating, LNPs can have a size in a range from about 10 nm to 500 nm, or from about 50 nm to about 200 nm, or from 75 nm to about 125 nm.

In certain embodiments concerning LNP, the LNP is described by Billingsley et al., Nano Lett. 2020, 20, 1578 or Billingsley et al., International Patent Publication No. WO 2021/077066 (both of which are hereby incorporated by reference herein in their entirety). Billingsley et al., and WO2021/077066 describe LNPs containing lipid-anchored PEG, cholesterol, phospholipid and ionizable lipids. In certain embodiments, the LNP contains a C14-4 polyamine core and/or has a particle size of about 70 nm. C14-4 has the following structure.

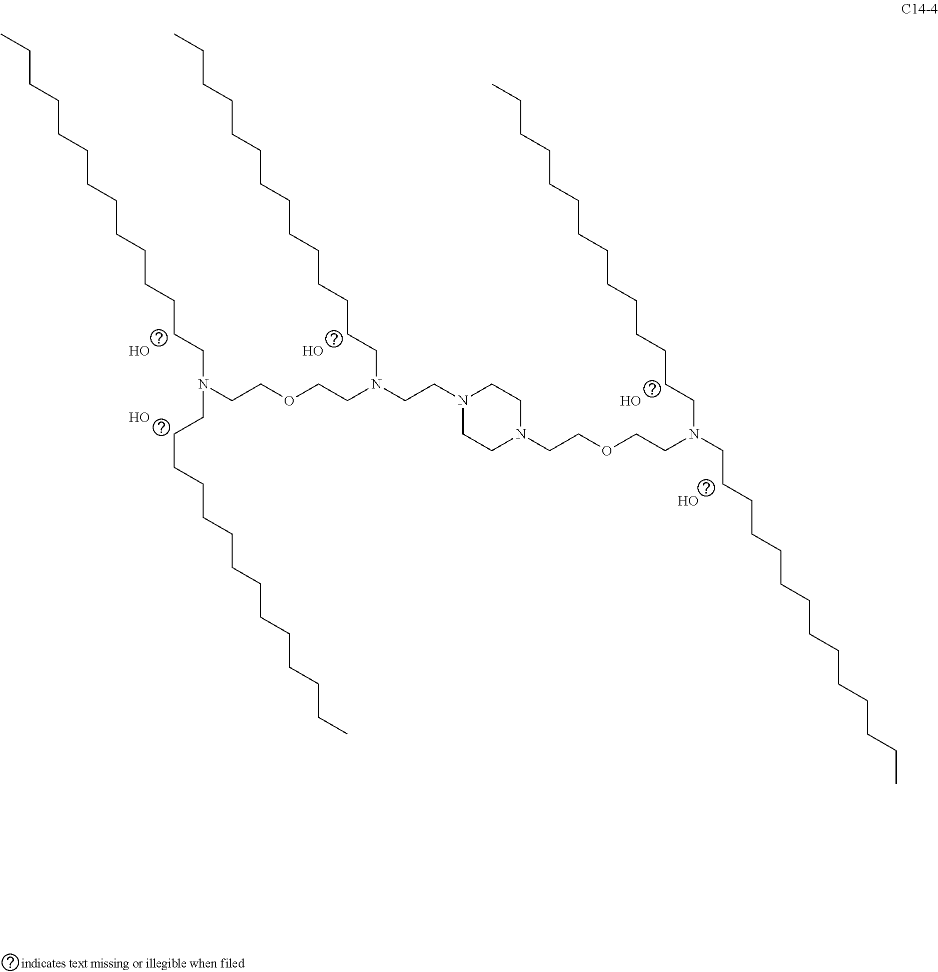

In certain embodiments the LNP is made up of a cationic lipid or lipopeptide described by U.S. Pat. Nos. 10,493,031, 10,682,374 or WO2021/077066 (each of which is hereby incorporated by reference herein in its entirety). In certain embodiments, the LNP contains a cationic lipid, a cholesterol-based lipid, and/or one or more PEG-modified lipids. In certain embodiments the LNP contains cKK-E12 (Dong et al., PNAS (2014) 111(11), 3955):

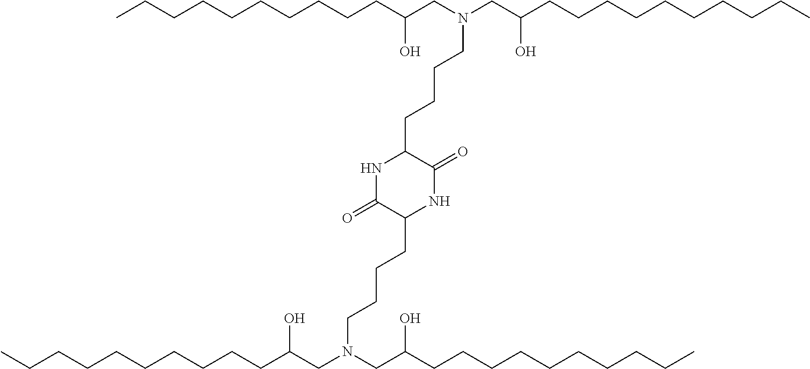 [SMILES: CCCCCCCCCCC(O)CN(CCCCC1NC(=O)C(CCCCN(CC(O)CCCCCCCCCC)CC(O)CCCCCCCCCC)NC1=O)CC(O)CCCCCCCCCC]

In certain embodiments the LNP comprises a modified form of cKK-E12 referred to herein as “bCKK-E12,” having the following structure:

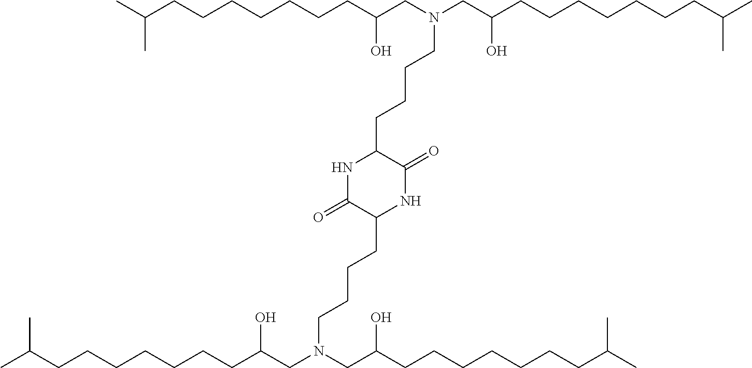 [SMILES: CC(C)CCCCCCCC(O)CN(CCCCC1NC(=O)C(CCCCN(CC(O)CCCCCCCC(C)C)CC(O)CCCCCCCC(C)C)NC1=O)CC(O)CCCCCCCC(C)C]

In certain embodiments the LNP comprises Lipid 1, 2, 3, 4, 5, 6, 7, 8, 9, or 10 as described by Sabnis et al., Molecular Therapy 2018, 26:6, 1509-1519 (hereby incorporated by reference herein in its entirety). In certain embodiments the LNP comprises Lipid 5, 8, 9, 10, or 11 described in Sabnis et al.

Lipid 5 of Sabnis et al. Has the Structure:

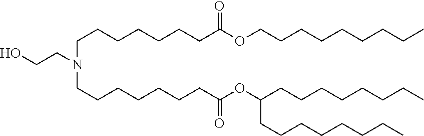 [SMILES: CCCCCCCCCOC(=O)CCCCCCCN(CCO)CCCCCCCC(=O)OC(CCCCCCCC)CCCCCCCC]

Lipid 9 of Sabnis et al. Has the Structure:

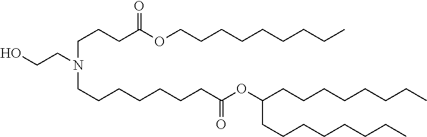 [SMILES: CCCCCCCCCOC(=O)CCCN(CCO)CCCCCCCC(=O)OC(CCCCCCCC)CCCCCCCC]

Additional lipids that may be utilized include those described by Roces et al., Pharmaceutics, 2020, 12,1095; Jayaraman et al, Angew. Chem. Int. Ed., 2012, 51, 8529-8533; Maier et al., www.moleculartherapy.org, 2013, Vol. 21, No. 8, 1570-1578; Liu et al., Adv. Mater. 2019, 31, 1902575, e.g., BAMEA-016B; Cheng et al., Adv. Mater., 2018, 30, 1805308, e.g., 5A2-SC8; Hajj and Ball, Small, 2019 15, 1805097, e.g., 306Oi10; Du et al., U.S. Patent Application Publication No. 2016/0376224; and Tanaka et al., Adv. Funct. Mater., 2020, 30, 1910575; each of which are hereby incorporated by reference herein in their entirety.

In certain embodiments, the LNP comprises mol % of the following components: one or more cationic lipids from about 20% to 65%, one or more phospholipid lipids from about 1% to about 50%, one or more PEG-conjugated lipid from about 0.1% to 10%, and cholesterol from about 0% to about 70%; one or more cationic lipids from about 20% to 50%, one or more phospholipid lipids from about 5% to about 20%, one or more PEG-conjugated lipid from about 0.1% to 5%, and cholesterol from about 20% to about 60%; in additional embodiments the phospholipid lipid is a neutral lipid; and the phospholipid lipid is DOPE or DSPC.

In certain embodiments the LNP, in mole %, comprises, consists essentially, or consists of the following components: (1) cKK-E12 (further described above and in Dong et al., PNAS (2014) 111(11), 3955), about 35%; C14-PEG2000, about 2.5%; cholesterol, about 46.5%; and DOPE, about 16%; or (2) Lipid 9 (Lipid 9 further described above and in Sabnis et al., (2018) Molecular Therapy 26:6, 1509-1519), about 50%; C14-PEG2000, about 1.5%; cholesterol, about 38.5%; and DSPC, about 10%.

In certain embodiment, the LNP, in mole %, comprises, consists essentially, or consists of the following components: bCKK-E12, about 35%; C14-PEG2000, about 2.5%; cholesterol, about 46.5%; and dioleoylphosphatidylethanolamine (DOPE), about 16%.

### I.B. Polymer-Based Nanoparticles

Polymer-based delivery systems can be made from a variety of different natural and synthetic materials. DNA and other compounds can be entrapped into the polymeric matrix of polymeric nanoparticles or can be adsorbed or conjugated on the surface of the nanoparticles. Examples of commonly used polymers for nucleic acid delivery include poly(lactic-co-glycolic acid) (PLGA), poly lactic acid (PLA), poly(ethylene imine) (PEI) and PEI derivatives, chitosan, dendrimers, polyanhydride, polycaprolactone, polymethacrylates, poly-L-lysine, pullulan, dextran, and hyaluronic acid, poly-$-aminoesters. (Thomas et al., (2019) Molecules 24, 3744.)

The polymeric-based nanoparticles can have different sizes, ranging from about 1 nm to about 1000 nm, from about 10 nm to about 500 nm, from about 50 nm to about 200 nm, from about 100 nm to about 150 nm, and from about 150 nm or less.

### I.C. Lipid Polymer Nanoparticles

Lipid polymer nanoparticles are hybrid nanoparticles providing both a lipid component and a polymer component, and as such can be considered to be an LNP or LPNP. The LPNP configuration can provide an outer polymer and inner lipid or an outer lipid and inner polymer. The presence of two different types of material facilitates designing nanoparticles to provide for delayed release of a component. Different lipid and polymer components can be selected taking into account the material be delivered. (For example, see Teo et al., Advanced Drug Delivery Reviews (2016) 98, 41; Bochicchio et al., Pharmaceutics (2021) 13, 198; Mahzabin and Das, IJPSR (2021) 12(1), 65; and Teixeira et al., (2017) Prog. Lipid Res. October; 68:1-11.)

### I.D. Protein and Peptide-Based Nanoparticles

Protein and peptide-based systems can employ a variety of different proteins and peptides. Examples of proteins include gelatin and elastin. Peptide-based systems can employ, for example, CPPs.

CPPs are short peptides (6-30 amino acid residues) potentially capable of intracellular penetration to deliver therapeutic molecules. The majority of CPPs consists mainly of arginine and lysine residues, making them cationic and hydrophilic, but CPPs can also be amphiphilic, anionic, or hydrophobic. CPPs can be derived from natural biomolecules (e.g., Tat, an HIV-1 protein), or obtained by synthetic methods (e.g., poly-L-lysine, polyarginine) (Singh et al., Drug Deliv. 2018; 25(1):1996-2006). Examples of CPPs include cationic CPPs (highly positively charged) such as the Tat peptide, penetratin, protamine, poly-L-lysine, and polyarginine; amphipathic CPPs (chimeric or fused peptides, constructed from different sources, containing both positively and negatively charged amino acid sequences), such as transportan, VT5, bactenecin-7 (Bac7), proline-rich peptide (PPR), SAP (VRLPPP)₃, TP10, pep-1, and MPG); membranotropic CPPs (exhibit both hydrophobic and amphipathic nature simultaneously, and comprise both large aromatic residues and small residues) such as H625, SPIONs-PEG-CPP and NPs; and hydrophobic CPPs (contain only non-polar motifs or residues) such as SG3, PFVYLI, pep-7, and fibroblast growth factors.

The protein and peptide nanoparticles can be provided in different sizes, for example, ranging from about 1 nm to about 1000 nm, from about 10 nm to about 500 nm, from about 50 nm to about 200 nm, from about 100 nm to about 150 nm, or from about 150 nm or less.

### I.E. Peptide Cage Nanoparticles

Peptide cage-based delivery systems can be produced from proteinaceous material able to assemble into a cage-like structure forming a constrained internal environment. Peptide cages can comprise a proteinaceous shell that self-assembles to form a protein cage (e.g., a structure with an interior cavity that is either naturally accessible to the solvent or can be made so by altering solvent concentration, pH, or equilibria ratios). The monomers of the protein cages can be naturally occurring or variant forms, including amino acid substitutions, insertions, and deletions (e.g., fragments).

Different types of protein “shells” can be assembled and loaded with different types of materials. Protein cages can be produced using viral coat protein(s) (e.g., from the Cowpea Chlorotic Mottle Virus protein coat), as well non-viral proteins (e.g., U.S. Pat. Nos. 6,180,389 and 6,984,386, U.S. Patent Application 20040028694, and U.S. Patent Application 20090035389, each of which is incorporated by reference herein in their entity).

Examples of protein cages derived from non-viral proteins include: eukaryotic or prokaryotic derived ferritins and apoferritins such as 12 and 24 subunit ferritins; and heat shock proteins (HSPs), such as the class of 24 subunit heat shock proteins that form an internal core space, the small HSP of Methanococcus jannaschii, the dodecameric Dsp HSP of E. coli; and the MrgA protein.

The protein cages can have different core sizes, such as ranging from about 1 nm to about 1000 nm, from about 10 nm to about 500 nm, from about 50 nm to about 200 nm, from about 100 nm to about 150 nm, or from about 150 nm or less.

### I.F. Exosomes

Exosomes are small biological membrane vesicles that been utilized to deliver various cargoes including small molecules, peptides, proteins and nucleic acids. Exosomes generally range in size from about 30 nm to 100 nm and can be taken up by a cell and deliver its cargo. Cargoes can be associated with exosome surface structure or may be encapsulated within the exosome bilayer.

Various modifications can be made to exosomes facilitating cargo delivery and cell targeting. Modifications for facilitating cargo delivery include structures for associating with cargoes such as protein scaffolds and polymers. Modifications for cell targeting include targeting ligands and modifying surface charge. Publications describing production, modification, and use of exosomes for delivery of different cargoes include Munagala et al., Cancer Leters (2021), 505, 58; Fu et al., (2020) Nanolmpact 20, 100261; and Dooley et al., (2021) Molecular Therapy 29(5), 1729 (each of which is hereby incorporated by reference herein).

### II. Infectious Disease Treatment

Infectious disease treatment refers to pathogens such as viruses, bacteria, fungi and parasites. The immune enhancement provided by nanoparticle delivery of dsDNA can help boost the immune system to attack the pathogen. In certain embodiments, nanoparticle delivery of dsDNA is used in combination with a therapeutic agent that can boost one or more components of the host immune system and/or target a particular pathogen; and with vaccine.

A variety of treatments can be employed to target pathogens including small molecules, proteins, polypeptides, antibodies, and nucleic acids. Nucleic acid treatment may include, for example, providing a transgene encoding an antigen, an immune component, a therapeutic protein, or a functional nucleic acid targeting a pathogen or pathogen component. Examples of functional nucleic acid targeting a pathogen nucleic acid includes a short hair pin RNA (shRNA), a small interfering RNA (siRNA), a microRNA (miRNA), a RNAi, a ribozyme, an antisense RNA, a clustered regularly interspaced short palindromic repeats (CRISPR)/Cas9 construct, a zinc finger nuclease (ZFN), and or a transcription activator-like effector nuclease (TALEN).

### II.A. Antiviral

Nanoparticle delivery of dsDNA can be used in treatments against different infectious virus. In certain embodiments the virus being treated is selected from the group consisting of: coronavirus (e.g., 229E, NL63, OC43, HKU1, MERS-CoV, SARS-CoV and SARS-CoV-2), Hepatitis C virus (HCV), Hepatitis B virus (HBV), Herpes simplex virus (HSV), Human immunodeficiency virus (HIV), Human papillomavirus (HPV), Influenza virus, Respiratory syncytial virus (RSV), Human cytomegalovirus (HCMV), and Varicella-zoster virus (VZV). In further embodiments, nanoparticle delivery of dsDNA is used in combination with an antiviral agent. Examples of FDA approved anti-viral agents for treating different viruses are provided in Rao et al., (2021) International Journal of Biological Macromolecules 172: 524-541 (hereby incorporated by reference herein in its entirety). Table 1 provides examples of FDA approved or authorized antiviral agents that may be used in combination with nanoparticle delivery of dsDNA.

TABLE 1

| Virus | Antiviral |
|---|---|
| Coronaviruses: | VEKLURY ® (remdesivir), PAXLOVID ™ (nirmatrelvir and ritonavir |
| 229E | tablets), REGEN-COV ® (casirivimab and imdevimab), sotrovimab, |
| NL63 | bamlanivimab, etesevimab, and molnupiravir |
| OC43 | |
| HKU1 | |
| MERS-CoV | |
| SARS-CoV | |
| SARS-CoV-2 | |
| HBV | Interferon alfa-2b (INT2B), lamivudine (3TC), adefovir dipivoxil (ADE), |
| | entecavir (ENT), peginterferon alfa-2a (PEG2a), telbivudine (LdT), |
| | tenofovir disoproxil fumarate (TDF), and tenofovir alafenamide fumarate |
| | (TAF) |
| HCMV | Ganciclovir sodium (GCV), foscarnet sodium (PFA), cidofovir (CID), |
| | fomivirsen sodium (FMV), valganciclovir hydrochloride (VALG), and |
| | letermovir (LET) |
| HCV | Simeprevir; sofosbuvir; ledipasvir/sofosbuvir; |
| | dasabuvir/ombitasvir/paritaprevir/ritonavir; daclatasvir/asunaprevir; |
| | ombitasvir/paritaprevir/ritonavir; daclatasvir/sofosbuvir; |
| | elbasvir/grazoprevir; sofosbuvir/valpatasvir; |
| | sofosbuvir/valpatasvir/voxilaprevir; and glecaprevir/pibrentasvir |
| HIV | Zidovudine (AZT), Didanosine (ddl), Zalcitabine (ddC), Stavudine (d4T), |
| | Lamivudine (3TC), Saquinavir mesylate (SQV), Indinavir sulfate (IDV), |
| | Ritonavir (RTV), Nevirapine (NVP), Lamivudine (3TC)/zidovudine |
| | (AZT), Delavirdine mesylate (DLV), Nelfinavir mesylate (NFV), |
| | Efavirenz (EFV), Abacavir sulfate (ABC), Amprenavir (APV), Lopinavir |
| | (LPV)/ritonavir (RTV), Abacavir sulfate (ABC)/lamivudine |
| | (3TC)/zidovudine (AZT), Tenofovir disoproxil fumarate (TDF), |
| | Emtricitabine (FTC), Enfuvirtide (T20), Fosamprenavir calcium (FPV), |
| | Atazanavir sulfate (ATV), Abacavir sulfate (ABC)/lamivudine (3TC), |
| | Emtricitabine (FTC)/tenofovir disoproxil fumarate (TDF), Tipranavir |
| | (TPV), Efavirenz (EFV)/emtricitabine (FTC)/tenofovir disoproxil |
| | fumarate, (TDF), Darunavir ethanolate (DRV), Raltegravir potassium |
| | (RAL), Maraviroc (MVC), Etravirine (ETR), Emtricitabine |
| | (FTC)/rilpivirine hydrochloride (RPV)/tenofovir, disoproxil fumarate |
| | (TDF), Rilpivirine hydrochloride (RPV), Cobicistat (COBI)/elvitegravir |
| | (EVG)/emtricitabine (FTC)/tenofovir, disoproxil fumarate (TDF), |
| | Dolutegravir sodium (DTG), Abacavir sulfate (ABC)/dolutegravir sodium |
| | (DTG)/lamivudine (3TC), Elvitegravir (EVG), Lamivudine |
| | (3TC)/raltegravir (RAL), Atazanavir sulfate (ATV)/cobicistat (COBI), |
| | Cobicistat (COBI)/elvitegravir (EVG)/emtricitabine (FTC)/tenofovir, |
| | alafenamide fumarate (TAF), Cobicistat (COBI)/darunavir ethanolate |
| | (DRV), Emtricitabine (FTC)/tenofovir alafenamide fumarate (TAF), |
| | Emtricitabine (FTC)/rilpivirine hydrochloride (RPV)/tenofovir, |
| | alafenamide fumarate (TAF), Dolutegravir (DTG)/rilpivirine (RPV), |
| | Bictegravir (BIC)/emtricitabine (FTC)/tenofovir alafenamide fumarate |
| | (TAF), Lamivudine (3TC)/tenofovir disoproxil fumarate (TDF), |
| | Doravirine (DOR)/lamivudine (3TC)/tenofovir disoproxil fumarate, |
| | (TDF), Doravirine (DOR), Efavirenz (EFV)/lamivudine (3TC)/tenofovir |
| | disoproxil fumarate (TDF), Efavirenz (EFV)/lamivudine (3TC)/tenofovir |
| | disoproxil fumarate (TDF), Darunavir (DRV)/cobicistat |
| | (COBI)/emtricitabine (FTC)/tenofovir, alafenamide (TAF), Ibalizumab- |
| | uiyk (IBA), and Dolutegravir (DTG)/lamivudine (3TC) |
| HSV | Idoxuridine (IDU), Vidarabine (VDR), Trifluridine (TFT), Acyclovir |
| | (ACY), Foscarnet sodium (PFA), Famciclovir (FAM), Valacyclovir |
| | hydrochloride (VAL), Penciclovir (PEN), Docosanol (DOC), Brivudine |
| | (BVDU), and Acyclovir/hydrocortisone (ACY) |
| HPV | Interferon alfa-2b (INT2B), Interferon alfa-N3 (INTN3), Podofilox |
| | (PDX), Imiquimod (IMQ), and Sinecatechins (SIN) |
| Influenza | Amantadine, Ribavirin (RBV), Rimantadine (RIM), Zanamivir (ZAN), |
| | Oseltamivir (OTV), Laninamivir octanoate (LO), Peramivir (PER), |
| | Favipiravir (FPV), and Baloxavir marboxil (BXM) |
| RSV | Ribavirin (RBV), RSV-IGIV and Palivizumab (PZ) |
| VSV | Vidarabine (VDR), Varicella-zoster immunoglobulin (VZIG), Acyclovir |
| | (ACY), Brivudine (BVDU), and Varicella-zoster immune globulin |
| | (VariZIG) |

### II.B. Antibacterial

Nanoparticle delivery of dsDNA can be used in treatments against different infectious bacteria. In certain embodiments, the bacteria, or bacterial disease being treated, is selected from brucellosis, campylobacter infections, cat-scratch disease, cholera, Escherichia coli infections, gonorrhea, klebsiella, enterobacter and serratia infections, legionella infections, meningococcal infections, pertussis, plague, pseudomonas infections, salmonella infections, shigellosis, typhoid fever, tularemia, anthrax, diphtheria, enterococcal infections, erysipelothricosis, listeriosis, nocardiosis, pneumococcal infections, tuberculosis, Mycobacterium tuberculosis, staphylococcal infections, streptococcal infections, bejel, yaws, pinta, leptospirosis, lyme disease, rat-bite fever, relapsing fever, syphilis, actinomycosis, bacteroides infections, botulism, clostridial infections, and tetanus.

In further embodiments, nanoparticle delivery of dsDNA is used in combination with one or more antibacterial agent; and one or more antibacterial agent is selected from aminoglycosides (e.g., amikacin, gentamicin, kanamycin, neomycin, netilmicin, tobramycin, paromomycin, and streptomycin), ansamycins (e.g., rifaximin), carbapenems (e.g., ertapenem, doripenem, imipenem/cilastatin, and meropenem); cephalosporins (e.g., cefadroxil, cefazolin, cephradine, cephapirin, cephalothin, cefalexin, cefaclor, cefoxitin, cefotetan, cefmetazole, cefonicid, loracarbet, cefprozil, cefuroxime, cefiime, cefdinir, cefditoren, cefotaxime, cefpodoxime, ceftazidime, ceftibuten, ceftizoxime, moxalactam ceftriaxone, cefepime, ceftaroline fosamil, and ceftobiprole); lincosamides (e.g., clindamycin and lincomycin): lipopeptides (e.g., daptomycin); glycopeptides (e.g., vancomycin, teicoplanin, telavancin, dalbavancin, and oritavancin); macrolides (e.g., azithromycin, erythromycin and clarithromycin, roxithromycin, telithromycin, spirmycin, and fidaxomicin); monobactams (e.g., aztreonam); nitrofurans (e.g., furazolidone and nitrofurantoin); oxazolidinones (linezolid, posizolid, radezolid and torezolid), penicillins (e.g., penicillin, amoxicillin, ampicillin, flucloxacillin, co-amoxiclav, flucloxacillin and phenoxymethylpenicillin), polypeptides (e.g., bacitracin, colistin, and polymyxin B); quinolones and fluoroquinolones (e.g., ciprofloxacin, enoxacin, fatifloxacin, gemifloxcin, lomefloxacin, moxifloxacin, nadifloxacin, nalidixic acid, norfloxacin, ofloxacin, and levofloxacin); sulfonamides (e.g., mafenide, sulfacetamide, sulfadiazine, sulfadimethoxine, sulfamethoxazole, sulfasalazine, sulfisoxazole, and trimethoprim-sulfamethoxazole); tigecycline; tetracyclines (e.g., tetracycline, doxycycline, demeclocycline, metacycline, minocycline, oxytetracycline and lymecycline); chloramphenicol, fusidic acid, nitrofurantoin, trimethoprim, clindamycin, sulfonamides and trimethoprim, metronidazole and tinidazole. Various combination can be used, for example, tuberculosis (TB) can be treated using the four-drug combination: 1) rifampin, 2) isoniazid, 3) pyrazinamide, and 4) ethambutol, or the three-drug regimen of bedaquiline, pretomanid and linezolid.

### II.C. Antifungal Agents

Nanoparticle delivery of dsDNA can be used in treatments against different fungal infections. Examples of fungal infections include aspergillosis, candidiasis, and mucormycosis; and examples of therapeutic agents that can be used to treat fungal infections include fluconazole, itraconazole, voriconazole, posaconazole, isavuconazole, caspofungin, micafungin, anidulafungin, amphotericin B, flucytosine, and anidulafungin (See, Houšt' et al., (2020) Metabolites 10, no. 3: 106, hereby incorporated by reference herein it its entirety).

### II.D. Antiparasitic Agents

Nanoparticle delivery of dsDNA can be used in treatments against different parasitic infections, such as protozoa, helminthic, nematodes, and cestodes. Examples of antiparasitic agents and uses of the antiparasitic agents include: albendazole (e.g., treating loiasis, filariasis, giardiasis, cysticercosis, toxocariasis, echinococcosis, and soil-transmitted helminthiases); amphotericin B (e.g., treating leishmaniasis); benznidazole (e.g., treating Trypanosoma cruzi); diethylcarbamazine (e.g., treating lymphatic filariasis, loiasis, tropical pulmonary eosinophilia, and onchocerciasis); mebendazole (e.g., treating echinococcosis, toxocariasis, and trichinellosis); metronidazole (e.g., treating amoebiasis and giardiasis); miltefosine (e.g., treating leishmaniasis); moxidectin (e.g., treating onchocerciasis); nifurtimox (e.g., treating trypanosomiasis); nitazoxanide (e.g., treating amoebiasis and giardiasis); oxamniquine (e.g., treating schistosomiasis); paromomycin (e.g., treating visceral leishmaniasis, giardiasis, intestinal amebiasis, and cryptosporidiosis); pentamidine (e.g., treating T. brucei gambiense and Pneumocystis carinii); pentavalent antimony (e.g., treating viceral, cutaneous, and mucocutaneous leishmaniasis); praziquantel (e.g., treating schistosomiasis, intestinal fluke infections, liver fluke infections, paragonimiasis, and cysticercosis); pyrimethamine (e.g., treating toxoplasmosis); surami (e.g., treating T. brucei gambiense HAT); sulfadiazine (e.g., toxoplasmosis); tinidazole (e.g., giardiasis, amebiasis, and trichomoniasis); and triclabendazole (e.g., treating fascioliasis and paragonimiasis). (See, Jatali and Zeitlinger (2020) Clinical Pharmacokinetics 59:827-847, hereby incorporated by reference herein in its entirety.)

### II.E. Checkpoint Inhibitors

Up-regulation of immune checkpoint molecules, such as PD-1 and CTLA4, on immune cells occur during acute infections, decreasing the host immune system. (Wykes and Lewin (2018) Nat. Rev. Immunol. 18(2): 91-104). Checkpoint inhibitors can be used to decrease the ability of an invading organism to turn down the host immune system. In addition, checkpoint inhibitors used in cancer treatment were found to provide a benefit in managing viral infection (Gambichler et al. (2020) J. ImmunoTherapy of Cancer 8; e001145.)

Different checkpoint inhibitors such as atezolizumab, avelumab, cemiplimab, dostarlimab, durvalumab, ipilimumab, nivolumab, and pembrolizumab have been approved for cancer indications. In certain embodiments, such inhibitors are utilized in the methods provided herein in the treatment of infectious diseases. Additional checkpoint inhibitors are undergoing clinical trials, and in certain embodiments are used in the methods provided herein to treat infectious disease. (See, for example, Darvin et al., Experimental & Molecular Medicine (2018) 50:165, hereby incorporated by reference herein in its entirety).

In certain embodiments, treatment of an infectious disease comprises administration of a checkpoint inhibitor as therapeutic agent. In further embodiments, the infectious disease is selected from coronavirus (such as SARS-CoV-2), HIV, HBV, SIV, HCV, influenza, TB, listeria, malaria, toxoplasma, and leishmania; the checkpoint inhibitor is an antibody targeting the PD-1/PD-L1 pathway; the checkpoint inhibitor is selected from atezolizumab, avelumab, cemiplimab, dostarlimab, durvalumab, ipilimumab, nivolumab, and pembrolizumab; and/or the checkpoint inhibitor is used in combination with an anti-infectious agent directly targeting the infectious organism (e.g., Table 1).

### II.F Vaccines

Nanoparticle delivery of dsDNA can be used in combination with a variety of different types of vaccines targeting infectious disease. Examples of different types of vaccines include live attenuated, killed whole organism, toxoid, subunit (e.g., purified protein, recombinant protein, polysaccharide, and peptide), virus-like particle, outer membrane vesicle, protein-polysaccharide conjugate, viral vector, nucleic acid, bacterial vectored, and antigen presenting cells. (Pollard and Bijker (2021) Nat Rev Immunol 21, 83-100.) In certain embodiments, the vaccine used in combination with nanoparticle delivery of dsDNA is selected from inactivated vaccines (e.g., hepatitis A, influenza and rabies); live-attenuated vaccines (e.g., measles, mumps, rubella, rotavirus, chickenpox, zoster, or yellow-fever); messenger RNA (mRNA) vaccines (e.g., SARS-CoV-2); subunit, recombinant, polysaccharide, and conjugate vaccines (e.g., Haemophilus influenzae type b, Hepatitis B, human papillomavirus, whooping cough (part of the DTaP combined vaccine), pneumococcal disease (e.g., polysaccharide and conjugated polysaccharide vaccines), meningococcal disease, and shingles; toxoid vaccines (i.e., diphtheria and tetanus); and viral vector vaccines (e.g., SARS-CoV-2, ebola, Zika, and influenza). Additional vaccines along with guidance concerning the use of different vaccines is provided in, for example, Plotkin's Vaccine 7^{th} Edition (2018) Edited by Plotkin et al.; and Epidemiology and Prevention of Vaccine-Preventable Disease (2021) 1₄*h Edition, hypertext transfer protocol://www.cdc.gov/vaccines/pubs/pinkbook/index.html, both of which are hereby incorporated by reference herein in their entirety.

In certain embodiments, the vaccine is (1) a peptide based vaccine, (2) a DNA vaccine or (3) an RNA vaccine. In certain embodiments, the nanoparticle delivery of dsDNA is used with a vaccine in combination with, one or more antiviral agents (e.g., the antiviral agents provided in Table 1), one or more antibacterial agents (e.g., the antibacterial agents provided in Section II.B supra) and/or one or more checkpoint inhibitor (e.g., atezolizumab, avelumab, cemiplimab, dostarlimab, durvalumab, ipilimumab, nivolumab, and pembrolizumab).

### III. Pharmaceutical Compositions

Appropriate pharmaceutical compositions can be selected based on the compound being administered and administration route. The pharmaceutical composition contains one or more active component along with a pharmaceutical acceptable carrier. Reference to “pharmaceutically” or “pharmaceutically acceptable” refers to non-toxic molecular entities suitable for administration and/or storage. Pharmaceutical compositions can comprise more than one therapeutically active agent.

Examples of pharmaceutically acceptable carriers include a non-toxic (in the amount used) solid, semi-solid or liquid filler, diluent, encapsulating material, or formulation. Guidance concerning formulations for small molecule, vaccines, proteins and antibodies can be found, for example, in Remington (2020) The Science and Practice of Pharmacy 23rd Edition; D'Amico et al., (2021) Drug Deliv. and Transl. Res. 11, 353-372; and Strickley and Lambert (2021) Journal of Pharmaceutical Sciences 110: 2590-2608.

The form of the pharmaceutical compositions, the route of administration, the dosage and the regimen depend upon the condition to be treated, such as the severity of the illness, the age, weight, and sex of the patient. Pharmaceutical compositions can be formulated for different modes of administration such as for topical, oral, intranasal, parenteral, intraocular, intravenous, intramuscular, or subcutaneous administration.

In an embodiment, the pharmaceutical composition contains a formulation capable of injection into a subject. Examples of injectable formulation components include isotonic, sterile, saline solutions (e.g., monosodium or disodium phosphate, sodium, potassium, calcium or magnesium chloride and mixtures of such salts), buffered saline, sugars (e.g., dextrose), and water for injection. Pharmaceutical compositions include dry, for example, freeze-dried compositions which upon addition, depending on the case, of sterilized water or physiological saline, permit the constitution of injectable solutions. The doses used for the administration can be adapted as a function of various parameters such as mode of administration, relevant pathology, and duration of treatment.

Other pharmaceutically acceptable forms include tablets or other solids for oral administration, including time-release capsules.

### IV. Administration and Treatment

Administration routes and treatment regimens can be selected based upon the chosen compound, pharmaceutical composition, and indication being treated. Administration routes include topical, oral, intranasal, parenteral, intraocular, intravenous, intramuscular, and subcutaneous administration. Guidance concerning formulations and administration for small molecules, vaccines, proteins and antibodies can be found, for example, in Remington (2020) The Science and Practice of Pharmacy 23rd Edition; D'Amico et al., (2021) Drug Deliv. and Transl. Res. 11, 353-372; and Strickley and Lambert (2021) Journal of Pharmaceutical Sciences 110: 2590-2608). Additional guidance can be found, for example, in product inserts for approved therapeutics (e.g., see Table 1.)

Preferred doses provide an effective amount to achieve a detectable effect. Generally, small molecules will be administered in a dose of between 0.0001 and 10 mg/kg, or 0.001 to 1 mg/kg body weight. Generally, the dose for large compounds such as antibodies and polypeptides may vary from about 10 ng/kg up to about 100 mg/kg of body weight, or about 1 mg/kg/day to 10 mg/kg/day.

An effective dose for dsDNA is sufficient to provide a detectable effect in the host immune system and should enhance vaccination or treatment. Generally, dsDNA will be administered in a range of 0.0001 mg/kg to 2 mg/kg. In certain embodiments the dsDNA will be administered in a range of 0.0001 to 0.001 mg/kg, 0.001 to 0.01 mg/kg, 0.01 to 0.1 mg/kg, or 0.1 to 2 mg/kg.

Reference to “treatment” or “treat” refers to both prophylactic, and therapeutic treatment of a patient having a disease or disorder. Prophylactic treatment provides a decreased likelihood of contracting a disease or disorder or decreasing the potential severity of a disease or disorder. Therapeutic treatment provides a clinical meaningful amelioration in at least one symptom or cause associated with a disease or disorder.

Thus, treatments may include administration to subjects at risk of contracting a disease or disorder, suspected to have contracted the disease or disorder, as well as subjects who are ill or have been diagnosed as suffering from a disease or disorder. Methods of treating pathogens are able to reduce the spread of the pathogen, reduce the number of pathogen, reduce the likelihood of infection, prevent infection and disease, and/or inhibit the growth of the pathogen. Methods of vaccination provide for an immune response able to target an invading pathogen and include, for example, a decreased likelihood or severity of a pathogen infection; prevention of infection and disease; and/or stimulation of macrophages, T cells, and/or B-cells (lead to antibody production including neutralizing antibodies).

The terms “ameliorate”, and “amelioration” refer to a detectable or measurable improvement in a disease or disorder symptom or an underlying cellular response. A detectable or measurable improvement includes a subjective or objective decrease, reduction, inhibition, suppression, limit or control in the occurrence, frequency, severity, progression, or duration of the disease or disorder, or complication caused by or associated with the disease or disorder, or an improvement in a symptom or an underlying cause or a consequence of the disease or disorder, or a reversal of the disease or disorder.

The terms “effective amount” and “sufficient amount” are that amount required to obtain a desired effect. An effective amount can be provided in single or multiple doses to achieve a therapeutic or prophylactic effect.

An effective amount can be administered alone or in combination with another therapeutic agent, compound, composition, treatment, protocol, or therapeutic regimen. The amount can be proportionally increased, for example, based on the need of the subject, type, status, and severity of the disease or disorder treated or side effects.

Administration of one or more therapeutic agents, and a nanoparticle comprising dsDNA can be together or separately. In certain embodiments, a therapeutic agent (e.g., checkpoint inhibitor, Table 1 compound, and/or vaccine) and a nanoparticle comprising dsDNA are administered at the same time; are administered within about 15 minutes, within about 30 minutes, within about 60 minutes, within about 2 hours, within about 4 hours, within about 6 hours, within about 12 hours, within about a day, within about 2 days, within about 3 days, within about 4 days, within about 5 days, within about a week or within about 2 weeks. In some cases, where administration is at the same time a composition can comprise both (i) a nanoparticle comprising DNA and (ii) one or more therapeutic agents (e.g., checkpoint inhibitor, Table 1 compound and/or vaccine); or (i) a nanoparticle comprising DNA and (ii) one or more therapeutic agents (e.g., checkpoint inhibitor, Table 1 compound and/or vaccine), may be provided as separate compositions.

### V. Kits

Further provided herein is a kit providing in separate containers at least: (a) an effective amount of nanoparticle comprising dsDNA; and (b) an effective amount of therapeutic agent or a vaccine. Kit components are further described, for example, in Sections I-IV supra. The kit may also provide a label with instructions for administration according to the methods described herein.

### VI. Additional Aspects and Embodiments

### Additional Aspects and Embodiments Include

A first aspect describes a method of treating an infectious disease in a subject comprising administering to the subject: (a) a nanoparticle comprising a double-stranded DNA (dsDNA), wherein the dsDNA comprises a double-stranded region; and (b) a vaccine or a therapeutic agent. Preferably, the dsDNA comprises a dsDNA region at least 45 base pairs in length.

Embodiment 1 further describes the first aspect, wherein the subject has a pathogenic infection. In further embodiments, the infection is bacterial infection, a viral infection, a fungal infection or a parasitic infection.

Embodiment 1a further describes the first aspect and Embodiment 1, wherein either (i) the nanoparticle is a lipid nanoparticle and the dsDNA is noncoding; (ii) the nanoparticle is a lipid nanoparticle and the dsDNA lacks a promoter operatively linked to a coding region for expression in said subject; (iii) said therapeutic agent is a checkpoint inhibitor; and/or (iv) at least two different therapeutic agents are provided.

Embodiment 2 further describes the first aspect and Embodiments 1 and 1a, wherein the method comprises administering the vaccine to the subject. In further embodiments, the vaccine is a live attenuated, killed whole organism, toxoid, subunit (e.g., purified protein, recombinant protein, polysaccharide, and peptide), virus-like particle, outer membrane vesicle, protein-polysaccharide conjugate, viral vector, nucleic acid, bacterial vectored, or antigen presenting cells; the vaccine comprises a protein antigen; the vaccine comprises a polysaccharide antigen; and the vaccine is a conjugate polysaccharide-polypeptide vaccine.

Reference to a particular embodiments includes reference to further embodiments provided therein. For example, reference in the second embodiment to the first embodiment provides a reference to all the embodiments provided in the first embodiment including the further embodiments provided therein.

Embodiment 3 further describes the first aspect and Embodiments 1, 1a and 2, wherein the vaccine is selected from hepatitis A, influenza and rabies inactivated vaccines; measles, mumps, rubella, rotavirus, chickenpox, zoster, or yellow-fever live-attenuated vaccines; SARS-CoV-2 mRNA vaccine; Haemophilus influenzae type b, Hepatitis B, human papillomavirus, DTaP, meningococcal disease, shingles, vaccine which is a subunit, recombinant, polysaccharide, polypeptide or conjugate vaccine; diphtheria and tetanus toxoid vaccine; and SARS-CoV-2, ebola, Zika, and influenza viral vector vaccine.

Embodiment 4 further describes the first aspect and any of Embodiments 1-3 (including 1a), wherein the method comprises administering a therapeutic agent. More than one type of therapeutic agent can be administered. In further embodiments, the therapeutic agent is a small molecule, protein, polypeptide, antibody or nucleic acid.

Embodiment 5 further describes the first aspect and any of Embodiments 1-4 (including 1a), wherein the therapeutic agent is an antiviral agent. In further embodiments, the subject is infected with a virus causing an infectious disease, the antiviral infection is as provided in Table 1, and/or the antiviral agent is as provided in Table 1.

Embodiment 6 further describes the first aspect and any of Embodiments 1-4 (including 1a), wherein the therapeutic agent is an anti-bacterial agent. In further embodiments, the subject is infected with a bacteria causing an infectious disease, the infectious disease is as provided in Section II.B. supra and/or the anti-bacterial agent is as described in Section II.B supra.

Embodiment 7 further describes the first aspect and any of Embodiments 1-4 (including 1a), wherein the therapeutic agent in an anti-fungal agent. In further embodiments, the subject is infected with a fungi causing an infectious disease, the infectious disease is as provided in Section II.C. supra and/or the anti-fungal agent is as described in Section II.C supra.

Embodiment 8 further describes the first aspect and any of Embodiments 1-4 (including ta), wherein the therapeutic agent in an anti-parasitic agent. In further embodiments, the subject is infected with a parasite causing a cause an infectious disease, the infectious disease is as provided in Section II.D. supra and/or the anti-parasitic agent is as described in Section II.D supra.

Embodiment 9 further describes the first aspect and any of Embodiments 1-4 (including 1a), wherein the therapeutic agent is a checkpoint inhibitor; the checkpoint inhibitor is selected from atezolizumab, avelumab, cemiplimab, dostarlimab, durvalumab, nivolumab, ipilimumab, and pembrolizumab; and/or the checkpoint inhibitor is an anti-PD-L1 antibody or an anti-PD-1 antibody.

Embodiment 10 further describes the first aspects and any of Embodiments 1-9 (including 1a), wherein at least two different therapeutic agents are administered. In further embodiments, one of therapeutic agents is a checkpoint inhibitor, and the checkpoint inhibitor is selected from atezolizumab, avelumab, cemiplimab, dostarlimab, durvalumab, nivolumab, ipilimumab, and pembrolizumab.

Embodiment 11 further describes the first aspect and any of Embodiments 1-10 (including 1a), wherein the vaccine and/or therapeutic agent are administered at, or about, the same time as the nanoparticle comprising the dsDNA.

A second aspect describes a method for enhancing an immune response to a vaccine in a subject, comprising administering to the subject (a) a nanoparticle comprising dsDNA; and (b) the vaccine. Preferably, the dsDNA comprises a dsDNA region at least 45 base pairs in length and/or the nanoparticle is a lipid nanoparticle.

Embodiment 12 further describes the second aspect, wherein the immune response is a T cell response. In a further embodiment, the T cell response is a Th1 or Th2 response.

Embodiment 13 further describes the second aspect and Embodiments 11 or 12, wherein the vaccine is a live attenuated, killed whole organism, toxoid, subunit (e.g., purified protein, recombinant protein, polysaccharide, and peptide), virus-like particle, outer membrane vesicle, protein-polysaccharide conjugate, viral vector, nucleic acid, bacterial vectored, or antigen presenting cells; the vaccine comprises a protein antigen; the vaccine comprises a polysaccharide antigen; and the vaccine is a conjugate polysaccharide-polypeptide vaccine.

Embodiment 14 further describes the first and aspects, and any of Embodiments 1-13 (including 1a), wherein the dsDNA comprises a dsDNA region at least 50 base pairs in length. In further embodiments: (1) the dsDNA region is at least 100 base pairs in length, at least 200 base pairs in length, at least 250 base pairs in length, at least 300 base pairs in length, at least 400 base pairs in length, at least 500 base pairs in length, at least 600 base pairs in length, at least 700 base pairs in length, at least 800 base pairs in length, at least 900 base pairs in length, at least 1000 base pairs in length, at least 1100 base pairs in length; at least 1200 base pairs in length, at least 1300 base pairs in length, at least 1400 base pairs in length, or at least 15,000 base pairs in length; and/or has a size range between any two of the mentioned sizes in (1).

Embodiment 15 further describes the first and second aspects and any of Embodiments 1-14 (including 1a), wherein the dsDNA contains 6 or fewer CpGs, 5 or fewer CpGs, 4 or fewer CpGs, 3 or fewer CpGs, 2 CpGs, 1 or fewer CpG, or zero CpG.

Embodiment 16 further describes the first and second aspects and any of Embodiments 1-15 (including 1a), wherein the dsDNA region and other nucleotides, if present, are naturally occurring and/or modified. In further embodiments, the dsDNA is modified and stimulates an innate immune response of at least 50%, at least 65%, at least 75%, at least 85%, at least 90%, or least 100% compared to the corresponding unmodified dsDNA as measured by IFN-γ and IL-6 as provided in the Examples below.

Embodiment 17 further describes the first and second aspects and any of Embodiments 1-16 (including 1a), wherein the nucleotides making up the dsDNA region and other nucleotides, if present, are unmodified or contain one or more modified nucleotides selected from the group consisting 2′-methoxyethyl (2′-MOE), 2′-fluor (2′-F), locked nucleic acid (LNA), constrained ethyl (cEt), tricyclo-DNA (tc-DNA), C7-modified deaza-adenine (methyl, Cl or F), C7-modified deaza-guanosine (methyl, Cl or F), C5-modified cytosine (methyl, F or Cl), and C5-modified uridine (methyl, F or Cl), and/or backbone modifications (phosphorothioate (Rp and/or Rs), thio-phosphoramidate, phosphorodiamidate morpholino oligos (PMO), and peptide-nucleic acid (PNA)). In further embodiments the one or more modifications are phosphorothioate (Rp and/or Rs). In further embodiment, the dsDNA is modified and stimulates an innate immune response of at least 50%, at least 65%, at least 75%, at least 85%, at least 90%, or least 100% compared to the corresponding unmodified dsDNA as measured by IFN-γ and IL-6 as provided in the Examples below.

Embodiment 18 further describes Embodiments 16 and 17, wherein no more than 95%, no more than 85%, no more than 75%, no more than 65%, no more than 55%, no more than 45%, no more than 35%, no more than 30%, no more than 25%, no more than 20%, no more than 15%, no more than 10%, no more than 5%, or 0% of the nucleotides are modified.

Embodiment 19 further describes Embodiments 16-18, wherein the dsDNA and/or dsDNA region (1) is at least 50 base pairs in length, at least 100 base pairs in length, at least 200 base pairs in length, at least 250 base pairs in length, at least 300 base pairs in length, at least 400 base pairs in length, at least 500 base pairs in length, at least 600 base pairs in length, at least 700 base pairs in length, at least 800 base pairs in length, at least 900 base pairs in length, at least 1000 base pairs in length, at least 1100 base pairs in length, at least 1200 base pairs in length, at least 1300 base pairs in length, at least 1400 base pairs in length, or at least 15,000 base pairs in length; and/or has a size range between any two of the mentioned sizes in (1); wherein no more than 95%, no more than 85%, no more than 75%, no more than 65%, no more than 55%, no more than 45%, no more than 35%, no more than 30%, no more than 25%, no more than 20%, no more than 15%, no more than 10%, no more 5%, or 0% of the dsDNA region is modified; where the remaining nucleotides, if present, may have the same percentage of modifications or different percentage of modifications as the dsDNA region in (1). In further embodiments no more than 95%, no more than 85%, no more than 75%, no more than 65%, no more than 55%, no more than 45%, no more than 35%, no more than 30%, no more than 25%, no more than 20%, no more than 15%, no more than 10%, no more 5% or 0% of the nucleotides outside of the dsDNA region in (1), if present, are modified.

Embodiment 20 further describes the first and second aspects and any of Embodiments 1-19 (including 1a), wherein the dsDNA region is formed by two separate polynucleotides or two regions of a single polynucleotide.

Embodiment 21 further describes the first and second aspects and any of Embodiments 1-19 (including 1a), wherein the dsDNA is linear or circular. In further embodiments, the dsDNA is selected from the group consisting of a minicircle, a plasmid, an open linear duplex DNA, and a closed-ended linear duplex DNA.

Embodiment 22 further describes the first and second aspects and any of Embodiments 1-21 (including 1a), wherein the dsDNA is noncoding, lacks a promoter coupled to a coding region for expression in the subject being treated (e.g., human cell) and/or is not a DNA vector comprising a transgene. Reference to “noncoding” indicates the dsDNA does not code for a gene (express a gene product) in the subject.

Embodiment 23 further describes the first and second aspects and any of Embodiments 1-22 (including 1a), wherein the nanoparticle is a lipid nanoparticle, polymeric nanoparticle, lipid polymer nanoparticles (LPNP), protein and peptide-based nanoparticles, DNA dendrimers or DNA-based nanocarriers, carbon nanotubes, microparticles, microcapsules, inorganic nanoparticle, or peptide cage nanoparticles; the nanoparticle is a LNP or LPNP; or the nanoparticle is an LNP, and the LNP in mole %, comprises, consists essentially, or consists of the following components (1) cKK-E12, about 35%; C14-PEG2000, about 2.5%; cholesterol, about 46.5%; and DOPE, about 16%; (2) Lipid 9, about 50%; C14-PEG2000, about 1.5%; cholesterol, about 38.5%; and DSPC, about 10%; or (3) or (3) bCKK-E12, about 35%; C14-PEG2000, about 2.5%; cholesterol, about 46.5%; and dioleoylphosphatidylethanolamine (DOPE), about 16%.

Embodiment 23a further describes the first and second aspects and any of Embodiments 1-22 (including 1a), wherein the nanoparticle comprises mol % the following components: (1) one or more cationic lipids from about 20% to 65%, one or more phospholipids from about 1% to about 50%, one or more PEG-conjugated lipids from about 0.1% to about 10%, and cholesterol from about 0% to about 70%; or (2) one or more cationic lipids from about 20% to about 50%, one or more phospholipids from about 5% to about 20%, one or more PEG-conjugated lipids from about 0.1% to about 5%, and cholesterol from about 20% to about 60%; in additional embodiments the phospholipid lipid is a neutral lipid; and the phospholipid lipid is DOPE or DSPC.

Embodiment 24 further describes the first and second aspects and any of Embodiments 1-23 (including 1a and 23a), wherein the subject is a human subject.

A third aspect describes a nanoparticle comprising dsDNA for use in the method of the first and second aspects and any of Embodiments 1-24 (including 1a and 23a). Preferably, wherein the dsDNA comprises a double-stranded region of at least 45 base pairs in length.

A fourth aspect describes the use of a nanoparticle comprising dsDNA for the preparation of a medicament. In different embodiments, the dsDNA comprises a double-stranded region of at least 45 base pairs in length; and/or the medicament is for use in the methods of the first and second aspects and any of Embodiments 1-24 (including 1a and 23a).

### EXAMPLES

Examples are provided below further illustrating different features of the present invention and methodology for practicing the invention. The provided examples do not limit the claimed invention.

### Example 1: Immune Stimulating Properties of Nanoparticle-Formulated DNA

Adjuvant properties of lipid nanoparticle-formulated DNA (DNA-LNP) were evaluated using SARS-CoV-2 receptor binding protein (RBD) and compared to different adjuvants (25 μL of Adju-Phos and 25 μL of AddaVax). The DNA was provided by a CpG-free plasmid that does not provide for gene expression in mammalian cells. LNP was made up of (mol %): CKK-E12, 35%; C14-PEG2000, 2.5%; cholesterol 46.5%; and dioleoylphosphatidylethanolamine (DOPE), 16%. Adju-Phos is described in Mold et al., (2016) Sci Rep. August 12; 6:31578. AddaVax is described in Ott et al., (2000). Methods in Molecular Medicine, Vol 42, 211-228. RBD was obtained from AcroBiosystems (Cat #: SPD-C52H3).

BALB/c male mice were dosed intramuscularly (50 μL/animal quad) at days 0, 15 and 30. Table 2 illustrates the study design (“DNA-NP” denotes the LNP-formulated DNA).

TABLE 2

| Group | n | antigen | | | adjuvant | | |
|---|---|---|---|---|---|---|---|
| 1 | 5 | — | — | | — | — | |
| 2 | 5 | RBD protein | 1 | μg | — | — | |
| 3 | 5 | — | — | | DNA-NP | 1 | μg |
| 4 | 5 | RBD protein | 1 | μg | Adju-Phos | | |
| 5 | 5 | | | | AddaVax | | |
| 6 | 5 | | | | DNA-NP | 0.1 | μg |
| 7 | 5 | | | | | 1 | μg |
| 8 | 5 | | | | DNA | 0.1 | μg |
| 9 | 5 | | | | | 1 | μg |
| 10 | 5 | RBD protein | 0.1 | μg | Adju-Phos | | |
| 11 | 5 | | | | AddaVax | | |
| 12 | 5 | | | | DNA-NP | 0.1 | μg |
| 13 | 5 | | | | | 1 | μg |
| 14 | 5 | | | | DNA | 0.1 | μg |
| 15 | 5 | | | | | 1 | μg |

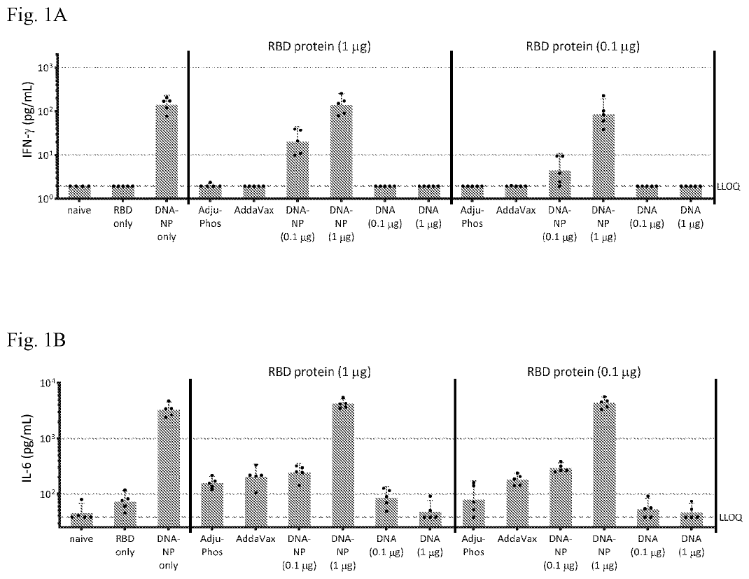

FIGS. 1A and 1B illustrate cytokine response in mice 6 hours post-dosing. FIG. 1A shows IFN-γ levels and FIG. 1B shows IL-6 levels.

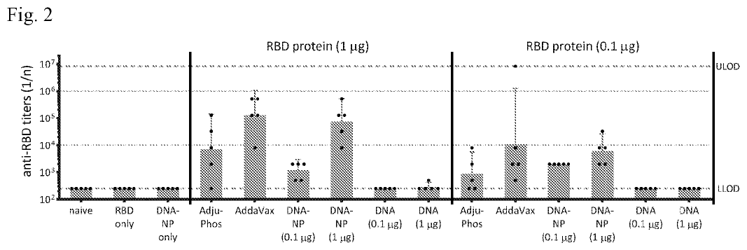

FIG. 2 illustrates IgG antibody titers in mice at day 38 post-dosing with COVID-19 receptor protein (RBD), DNA-LNP, and different adjuvants (Adju-Phos and AddaVax). Antibody titers were measured using anti-RBD ELISA.

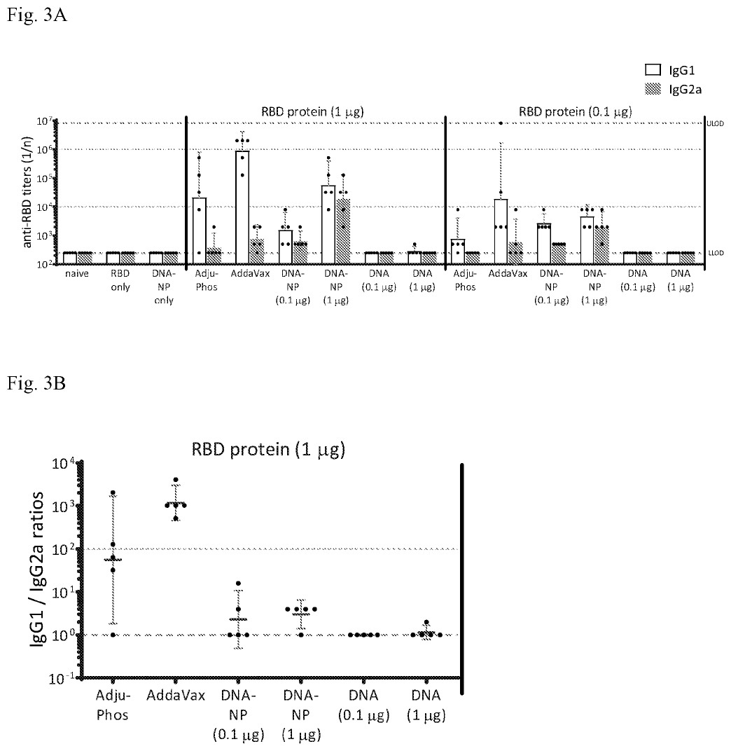

FIG. 3A and FIG. 3B illustrate IgG subtypes in mice at day 38 post-dosing with COVID-19 receptor protein (RBD), DNA-LNP, and different adjuvants (Adju-Phos and AddaVax).

DNA-LNP induced significantly higher IFN-γ and IL-6 levels in blood. Both DNA-NP and AddaVax demonstrated significant adjuvant potency. IgG isotype quantitation in mice is a proxy measurement for Th1 or Th2 immune responses. (Rostamian et al., (2017) (50) 160-166.) DNA-LNP induced a Th1 response different from that of AddaVax. A Th1 response is advantageous with prophylactic vaccines for generating both B-cells (to prevent infection) and T-cells (to fight off infection once it has occurred) in the immune response.

### Example 2: Immune Stimulating Properties of DNA-LNP

Adjuvant properties of DNA-LNP were evaluated using adeno-associated viral particles (AAV) as antigen. The DNA was provided by an approximately 1.3 kb plasmid that does not provide for gene expression in mammalian cells. LNP was made up of (mol %): bCKK-E12, 35%; C14-PEG2000, 2.5%; cholesterol 46.5%; and dioleoylphosphatidylethanolamine (DOPE), 16%.

C57BL/6 female mice were dosed intravenously with AAV with 1×10¹² vector genomes (vg) per animal at day 0. Some mice received AAV mixed with DNA-LNP (5 μg) or AAV mixed with “empty” LNP (no DNA) (dose equivalent to the amount of LNP in 5 μg of DNA-LNP) intravenously. Plasma was collected to analyze anti-AAV IgG and cytokine levels.

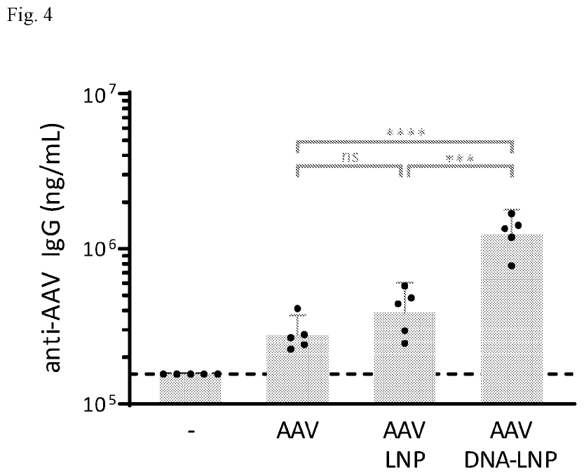

FIG. 4 illustrates anti-AAV antibody levels in mice at two weeks after dosing with AAV, AAV mixed with “empty” LNP (no DNA), or AAV adjuvanted with DNA-LNP. AAV mixed with DNA-LNP increased the level of anti-AAV IgG in plasma.

Table 3 illustrates cytokine levels in plasma 4 hour after dosing AAV, AAV mixed with “empty” LNP, or AAV mixed with DNA-LNP. Cytokine level was increased by the dosing of AAV mixed with DNA-LNP. Cytokine levels were increased by the administration of AAV mixed with DNA-LNP. AAV and empty LNP showed no adjuvant effect.

TABLE 3

| | AAV | | AAV + LNP | | AAV + DNA-LNP | |
|---|---|---|---|---|---|---|
| pg/mL | average | SD | average | SD | average | SD |
| IFN- α | 20.9 | 0.0 | 20.9 | 0.0 | 5601.1 | 1079.5 |
| IFN-β | 40.0 | 0.0 | 40.0 | 0.0 | 10282.7 | 6611.9 |
| IFN-γ | 5.5 | 0.0 | 10.8 | 10.5 | 2616.3 | 1686.1 |
| IL-6 | 27.3 | 0.0 | 35.0 | 15.4 | 43880.1 | 29590.4 |
| KC/GRO | 8.1 | 0.0 | 10.3 | 4.4 | 1281.6 | 339.1 |
| MCP-1 | 60.1 | 0.0 | 60.1 | 0.0 | 12815.8 | 1927.0 |
| MIP-1β | 5.8 | 1.0 | 10.5 | 4.4 | 3074.7 | 2346.3 |
| TNF- α | 14.3 | 0.0 | 15.2 | 1.9 | 1364.1 | 1810.5 |
| IL-18 | 149.8 | 0.0 | 264.7 | 101.4 | 4445.9 | 844.8 |

### Example 3: Immune Signaling Pathway

Cytokine levels after DNA-LNP administration was evaluated in C57BL/6 knockout (KO) mice deficient in different immune signaling pathways. DNA-LNP composition is as provided in Example 1. Mice were dosed intravenously with DNA-LNP (5 μg). Plasma was collected 3 or 4 hours after DNA-LNP dosing for cytokine analyses.

The results illustrate the immune signaling pathways contributing to the cytokine levels induced after DNA-LNP administration. Cytokine levels in wild type mice (black line) and multiple KO mice (gray line) were compared: cGAS KO (Table 4), STING KO (Table 4), TLR9 KO (Table 5), IFNAR KO (Table 5); AIM2 KO (Table 6), MyD88 KO (Table 6); RIG-I KO (Table 7), NLRP3 KO (Table 8), Caspase 1 KO (Table 9); and Gasdermin D KO (Table 10). cGAS and STING contribute to the cytokine levels but did not affect the level of IL-18, which is a cytokine induced by the inflammasome pathway. The results from the knock mice show that inflammasome mediators, such as AIM2, Caspase 1, and Gasdermin D contributed to the induction of IL-18 and IFN-γ produced by DNA-LNP administration.

TABLE 4

| | baseline | wild type | | cGAS KO | | STING KO | |
|---|---|---|---|---|---|---|---|
| pg/mL | value | average | SD | average | SD | average | SD |
| IFN- α | 154.0 | 27059.0 | 13071.3 | 154.3 | 0.0 | 154.3 | 0.0 |
| IFN-β | 4.2 | 2453.6 | 1295.4 | 4.2 | 0.0 | 4.2 | 0.0 |
| IFN-γ | 0.5 | 1580.4 | 941.5 | 20.0 | 10.6 | 15.8 | 3.2 |
| IL-6 | 49.4 | 21944.3 | 11713.9 | 206.1 | 146.6 | 532.5 | 205.1 |
| KC/GRO | 189.0 | 838.2 | 328.3 | 1223.2 | 644.0 | 2025.8 | 548.6 |
| MCP-1 | 32.7 | 11304.9 | 4582.1 | 433.3 | 277.8 | 455.6 | 234.0 |
| MIP-1β | 52.7 | 14661.8 | 6071.5 | 156.3 | 62.8 | 204.9 | 84.7 |
| TNF- α | 14.0 | 635.9 | 266.2 | 10.9 | 2.0 | 12.6 | 1.4 |
| IL-18 | 354.2 | 1929.8 | 1284.9 | 2801.8 | 1954.1 | 3316.2 | 941.8 |

TABLE 5

| | baseline | wild type | | TLR9 KO | | IFNAR KO | |
|---|---|---|---|---|---|---|---|
| pg/mL | value | average | SD | average | SD | average | SD |
| IFN- α | 154.0 | 27059.0 | 13071.3 | 27624.7 | 5894.4 | 6783.6 | 1688.4 |
| IFN-β | 4.2 | 2453.6 | 1295.4 | 3486.7 | 3012.2 | 2960.9 | 321.1 |
| IFN-γ | 0.5 | 1580.4 | 941.5 | 2701.4 | 599.7 | 36.7 | 8.2 |
| IL-6 | 49.4 | 21944.3 | 11713.9 | 42228.5 | 34135.1 | 3942.7 | 1573.2 |
| KC/GRO | 189.0 | 838.2 | 328.3 | 1741.6 | 1451.8 | 3815.3 | 1082.6 |
| MCP-1 | 32.7 | 11304.9 | 4582.1 | 13632.2 | 1437.9 | 4349.4 | 559.7 |
| MIP-1β | 52.7 | 14661.8 | 6071.5 | 19373.4 | 11452.1 | 4871.8 | 755.8 |
| TNF- α | 14.0 | 635.9 | 266.2 | 1037.9 | 796.2 | 298.7 | 53.2 |
| IL-18 | 354.2 | 1929.8 | 1284.9 | 2387.6 | 546.0 | 1804.4 | 413.2 |

TABLE 6

| | baseline | wild type | | AIM2 KO | | MyD88 KO | |
|---|---|---|---|---|---|---|---|
| pg/mL | value | average | SD | average | SD | average | SD |
| IFN-α | 222.3 | 53705.6 | 3425.7 | 50028.9 | 4976.0 | 41330.1 | 6665.1 |
| IFN-β | 6.3 | 2537.0 | 549.6 | 1329.2 | 276.6 | 3219.8 | 1271.9 |
| IFN-γ | 0.8 | 10027.0 | 2488.5 | 91.0 | 17.8 | 113.4 | 67.7 |
| IL-6 | 7.8 | 73633.5 | 13288.7 | 12108.4 | 1407.4 | 16502.9 | 4903.1 |
| KC/GRO | 132.8 | 2289.6 | 537.1 | 1846.8 | 217.4 | 556.4 | 141.5 |
| MCP-1 | 23.5 | 19668.0 | 408.3 | 16421.8 | 1655.6 | 19033.1 | 1449.9 |
| MIP-1β | 31.5 | 9849.5 | 760.0 | 7828.3 | 1657.6 | 10205.5 | 2389.7 |
| TNF-α | 9.1 | 996.7 | 363.3 | 464.0 | 36.1 | 829.5 | 301.0 |
| IL-18 | 144.4 | 6176.9 | 3101.5 | 330.5 | 51.0 | 5538.7 | 1046.6 |

TABLE 7

| | baseline | wild type | | RIG-I KO | |
|---|---|---|---|---|---|
| pg/mL | value | average | SD | average | SD |
| IFN-α | 338.4 | 36868.1 | 7154.2 | 23474.2 | 7908.4 |
| IFN-β | 7.6 | 3627.3 | 757.9 | 1761.8 | 542.6 |
| IFN-γ | 1.1 | 2112.2 | 705.4 | 694.1 | 527.1 |
| IL-6 | 14.2 | 40576.2 | 7257.5 | 16861.5 | 9217.0 |
| KC/GRO | 99.7 | 1627.9 | 597.6 | 1287.9 | 586.4 |
| MCP-1 | 28.5 | 3050.0 | 0.0 | 3050.0 | 0.0 |
| MIP-1β | 43.1 | 15910.6 | 801.1 | 11644.2 | 951.4 |
| TNF-α | 7.0 | 723.7 | 151.2 | 644.9 | 139.4 |
| IL-18 | 156.4 | 3294.6 | 776.9 | 1482.3 | 730.4 |

TABLE 8

| | baseline | wild type | | NLRP3 KO | |
|---|---|---|---|---|---|
| pg/mL | value | average | SD | average | SD |
| IFN-α | 397.3 | 44955.5 | 4375.7 | 24106.3 | 6717.5 |
| IFN-β | 15.1 | 4891.2 | 1266.8 | 1642.8 | 717.7 |
| IFN-γ | 1.4 | 2839.4 | 1228.8 | 1130.3 | 812.8 |
| IL-6 | 16.4 | 36661.8 | 6388.4 | 14871.4 | 4497.6 |
| KC/GRO | 145.2 | 2979.1 | 962.3 | 1617.0 | 698.1 |
| MCP-1 | 41.4 | 9.8 | 0.0 | 9.8 | 0.0 |
| MIP-1β | 65.2 | 17504.6 | 1436.7 | 9050.1 | 2434.4 |
| TNF-α | 8.3 | 1637.1 | 146.4 | 659.4 | 231.4 |
| IL-18 | 216.2 | 3954.5 | 1078.1 | 2448.1 | 880.7 |

TABLE 9

| | baseline | wild type | | Caspase-1 KO | |
|---|---|---|---|---|---|
| pg/mL | value | average | SD | average | SD |
| IFN-α | 338.4 | 34184.2 | 3665.9 | 26776.1 | 3453.3 |
| IFN-β | 7.6 | 2357.2 | 327.3 | 1458.4 | 159.0 |
| IFN-γ | 1.1 | 2381.7 | 510.0 | 31.5 | 8.9 |
| IL-6 | 14.2 | 41571.1 | 6226.1 | 8470.7 | 2255.2 |
| KC/GRO | 99.7 | 1510.2 | 391.7 | 795.0 | 54.5 |
| MCP-1 | 28.5 | 3050.0 | 0.0 | 3050.0 | 0.0 |
| MIP-1β | 43.1 | 12779.5 | 698.4 | 10320.8 | 1394.6 |
| TNF-α | 7.0 | 709.7 | 114.6 | 438.9 | 63.7 |
| IL-18 | 128.8 | 3275.8 | 590.8 | 128.0 | 0.0 |

TABLE 10

| | baseline | wild type | | GSDMD KO | |
|---|---|---|---|---|---|
| pg/mL | value | average | SD | average | SD |
| IFN-α | 316.0 | 72389.8 | 9211.6 | 51409.0 | 5402.2 |
| IFN-β | 33.3 | 8332.0 | 3978.6 | 5926.1 | 2222.9 |
| IFN-γ | 5.1 | 4569.5 | 2115.7 | 222.6 | 121.2 |
| IL-6 | 15.0 | 53000.0 | 0.0 | 27457.0 | 13392.6 |
| KC/GRO | 193.2 | 4406.2 | 1487.4 | 1439.2 | 999.5 |
| MCP-1 | 21.5 | 2975.0 | 0.0 | 2975.0 | 0.0 |
| MIP-1β | 52.7 | 38571.6 | 12349.6 | 26922.1 | 3292.6 |
| TNF-α | 6.7 | 2125.7 | 2005.9 | 941.5 | 170.3 |
| IL-18 | 148.9 | 2796.8 | 409.4 | 187.6 | 68.6 |

### Example 4: Immune Stimulating Properties of dsDNA with Different LNPs

Cytokine levels after DNA-LNP administration was evaluated in C57BL/6 mice. DNA-LNPs (25 g) comprised of different LNPs were dosed intravenously. The DNA was provided by a CpG-free plasmid that does not provide for gene expression in mammalian cells. LNP1 was made up of (mol %): bCKK-E12, 35%; C14-PEG2000, 2.5%; cholesterol 46.5%; and dioleoylphosphatidylethanolamine (DOPE), 16%. LNP2 was made up of (mole %): Lipid 9, ˜50% (Lipid 9 is further described in Section I.A.supra and Sabnis et al., (2018) Molecular Therapy 26:6, 1509-1519); ˜C14-PEG2000, 1.5%; cholesterol, ˜38.5%; and distearoylphosphatidylcholine (DSPC), ˜10%. LNP3 was GenVoy-ILM™ LNP (Precision NanoSystems). Roces et al., Pharmaceutics, 2020, 12,1095, indicates GenVoy-ILM™ LNP contains: ionizable lipid, about 50%; DSPC, about 10%; cholesterol, about 37.5%; and stabilizer (PEG-Lipid), about 2.5%.

Table 11 provides results on the cytokine levels induced 4 hours after various DNA-LNP administration.

TABLE 11

| | baseline | DNA-LNP1 | | DNA-LNP2 | | DNA-LNP3 | |
|---|---|---|---|---|---|---|---|
| pg/mL | value | average | SD | average | SD | average | SD |
| IFN-α | 822.6 | 62884.5 | 10799.3 | 86183.9 | 21185.1 | 4519.6 | 3110.0 |
| IFN-β | 29.8 | 3545.8 | 1618.2 | 3593.5 | 1449.8 | 287.4 | 151.8 |
| IFN-γ | 4.0 | 13912.3 | 5516.9 | 9831.0 | 2563.8 | 279.3 | 174.3 |
| IL-6 | 83.4 | 163879.4 | 25922.0 | 175874.9 | 12047.8 | 124430.2 | 32827.0 |
| KC/GRO | 446.9 | 14887.4 | 5575.6 | 21600.0 | 0.0 | 21600.0 | 0.0 |
| MCP-1 | 167.3 | 12200.0 | 0.0 | 12200.0 | 0.0 | 10163.5 | 1316.5 |
| MIP-1β | 196.3 | 22750.6 | 7330.8 | 36731.4 | 11319.8 | 7318.7 | 3296.3 |
| TNF-α | 14.1 | 1051.5 | 142.9 | 1131.7 | 282.6 | 210.8 | 56.3 |
| IL-18 | 1280.0 | 7813.3 | 1533.3 | 10088.3 | 2030.8 | 50000.0 | 0.0 |

A number of different aspects and embodiments of the instant invention have been described throughout the application. Nevertheless, the skilled artisan without departing from the spirit and scope of the instant invention, can make various changes and modifications of the instant invention to adapt it to various usages and conditions.

I/We claim:

1. A method of treatment for an infectious disease in a subject comprising administering to said subject:
a) a nanoparticle comprising a double-stranded DNA (dsDNA), wherein said dsDNA comprises a double-stranded region of at least 45 base pairs in length; and
b) a vaccine or a therapeutic agent;
wherein either (i) the nanoparticle is a lipid nanoparticle and the dsDNA is noncoding; (ii) the nanoparticle is a lipid nanoparticle and the dsDNA lacks a promoter operatively linked to a coding region for expression in said subject; (iii) said therapeutic agent is a checkpoint inhibitor; and/or (iv) at least two different therapeutic agents are provided.

2. The method of claim 1, wherein said nanoparticle is a lipid nanoparticle and the dsDNA is noncoding.

3. The method of claim 1, wherein said nanoparticle is a lipid nanoparticle and the dsDNA lacks a promoter operatively linked to a coding region for expression in said subject.

4. The method of any one of claims 1-3, wherein said therapeutic agent is a checkpoint inhibitor.

5. The method of claim 4, wherein said checkpoint inhibitor is an anti-PD-L1 antibody or an anti-PD-1 antibody.

6. The method of claim 5, wherein said checkpoint inhibitor is selected from the group consisting of: atezolizumab, avelumab, cemiplimab, dostarlimab, durvalumab, nivolumab, ipilimumab, and pembrolizumab.

7. The method of any one of claims 1-3, wherein said method comprises administering said therapeutic agent and said therapeutic agent is an anti-viral agent, anti-bacterial agent, anti-fungal agent, or an anti-parasitic agent.

8. The method of any one of claims 1-7, wherein said method comprises administering said vaccine, wherein said vaccine comprises a polypeptide antigen, a polysaccharide antigen or is a conjugated polysaccharide-polypeptide.

9. The method of any one of claims 1-8, wherein said subject has a pathogenic infection.

10. The method of any one of claims 1-9, wherein at least two different therapeutic agents are administered.

11. The method of claim 10, wherein the two different therapeutic agents comprise a first therapeutic agent select from an antiviral agent provided in Table 1 and a second therapeutic agent selected from atezolizumab, avelumab, cemiplimab, dostarlimab, durvalumab, nivolumab, ipilimumab, and pembrolizumab.

12. The method of any one of claims 1-11, wherein said vaccine and/or therapeutic agent are administered at, or about, the same time as said dsDNA.

13. A method for enhancing an immune response to a vaccine in a subject, comprising administering to said subject:
a) a nanoparticle comprising a double-stranded DNA (dsDNA), wherein said dsDNA comprises a double-stranded region of at least 45 base pairs in length, and
b) said vaccine, wherein the nanoparticle is a lipid nanoparticle.

14. The method of claim 13, wherein said immune response is a T cell response.

15. The method of claim 14, wherein said T cell response is a Th1 or Th2 response.

16. The method of any one of claims 13-15, wherein said vaccine comprises a polypeptide antigen, a polysaccharide antigen or is a conjugated polysaccharide-peptide.

17. The method of any one of claims 1-16, wherein said dsDNA region is at least 50 base pairs in length.

18. The method of claim 17, wherein said dsDNA region is at least 100 base pairs in length.

19. The method of claim 18, wherein said dsDNA is at least 200 base pairs in length.

20. The method of any one of claims 1-19, wherein said dsDNA is unmodified.

21. The method of any one of claims 1-20, wherein said dsDNA is linear or circular.

22. The method of any one of claims 1-20, wherein said dsDNA is selected from the group consisting of a minicircle, a plasmid, an open linear duplex DNA, and a closed-ended linear duplex DNA.

23. The method of any one of claims 1-20, wherein said dsDNA region is provided by two regions of a polynucleotide and said polypeptide comprises a loop region.

24. The method of any one of claims 13-23, wherein said dsDNA is noncoding, or lacks a promoter operatively linked to a coding region for expression in said subject.

25. The method of any one of claim 1-24, where said nanoparticle comprises mol % one or more cationic lipids from about 20% to about 50%, one or more phospholipids from about 5% to about 20%, one or more PEG-conjugated lipids from about 0.1% to about 5%, and cholesterol from about 20% to about 60%.

26. The method of any one of claims 1-24, wherein said nanoparticle comprises mol % one or more cationic lipids from about 20% to about 65%, one or more phospholipid from about 1% to about 50%, one or more PEG-conjugated lipid from about 0.1% to about 10%, and cholesterol from about 0% to about 70%.

27. The method of claims 25 or 26, wherein the phospholipid lipid is 1,2-dioleoyl-sn-glycero-3-phosphatidyl-ethanolamine or 1,2-distearoyl-sn-glycero-3-phosphocholine.

28. The method of claim 25, wherein said nanoparticle in mole %, comprises (1) cKK-E12, about 35%; C14-PEG2000, about 2.5%; cholesterol, about 46.5%; and 1,2-dioleoyl-sn-glycero-3-phosphatidyl-ethanolamine (DOPE), about 16%; or (2) Lipid 9, about 50%; C14-PEG2000, about 1.5%; cholesterol, about 38.5%; and 1,2-distearoyl-sn-glycero-3-phosphocholine (DSPC), about 10%.

29. The method of any one claims 1-3 or 13-15, wherein said nanoparticle comprises mol % one or more cationic lipids from about 20% to 50%, one or more phospholipids from about 5% to about 20%, one or more PEG-conjugated lipid from about 0.1% to 5%, and cholesterol from about 20% to about 60%.

30. The method of any one of claims 1-3 or 13-15, wherein said nanoparticle comprises mol % one or more cationic lipids from about 20% to 65%, one or more phospholipid lipids from about 1% to about 50%, one or more PEG-conjugated lipid from about 0.1% to 10%, and cholesterol from about 0% to about 70%.

31. The method of claim 30, wherein said phospholipid lipid is 1,2-dioleoyl-sn-glycero-3-phosphatidyl-ethanolamine or 1,2-distearoyl-sn-glycero-3-phosphocholine.

32. The method of claim 30, wherein said phospholipid lipid is 1,2-dioleoyl-sn-glycero-3-phosphatidyl-ethanolamine or 1,2-distearoyl-sn-glycero-3-phosphocholine.

33. The method of any one of claims 1-3 or 13-15 wherein said nanoparticle in mole %, comprises (1) cKK-E12, about 35%; C14-PEG2000, about 2.5%; cholesterol, about 46.5%; and 1,2-dioleoyl-sn-glycero-3-phosphatidyl-ethanolamine (DOPE), about 16%; or (2) Lipid 9, about 50%; C14-PEG2000, about 1.5%; cholesterol, about 38.5%; and 1,2-distearoyl-sn-glycero-3-phosphocholine (DSPC), about 10%.

34. The method of any one of claims 1-33, wherein said subject is a human.

35. A nanoparticle comprising dsDNA for use in the method of any one of claims 1-34, wherein said dsDNA comprises a double-stranded region of at least 45 base pairs in length.

36. Use of a nanoparticle comprising dsDNA for the preparation of a medicament, for the method of any one of claims 1-34, wherein said dsDNA comprises a double-stranded region of at least 45 base pairs in length.
